(19) United States
(12) Patent Application Publication
(10) Pub. No.: US 2022/0106585 A1
(43) Pub. Date: Apr. 7, 2022

(54) METHOD OF SEQUENCING NUCLEIC ACID WITH UNNATURAL BASE PAIRS

(71) Applicant: Agency for Science, Technology and Research, Singapore (SG)

(72) Inventors: Ichiro Hirao, Singapore (SG); Michiko Hirao, Singapore (SG); Kiyofumi Hamashima, Singapore (SG)

(21) Appl. No.: 17/427,576

(22) PCT Filed: Dec. 4, 2019
(86) PCT No.: PCT/SG2019/050597

§ 371 Date: Jul. 30, 2021

(30) Foreign Application Priority Data
Jan. 31, 2019 (SG) 10201900941T

(51) Int. Cl. C12N 15/10; C12Q 1/6874; C12Q 1/6806

(52) U.S. Cl. CPC C12N 15/1048; C12N 2320/13; C12Q 1/6806; C12Q 1/6874

(57) ABSTRACT

Disclosed is a method of sequencing a nucleic acid containing an unnatural base pair (UBP), comprising performing two or more replacement replication reactions wherein the nucleic acid is replicated using two or more intermediate of the unnatural base pair; sequencing the nucleic acid resulting from the replacement replication reactions; clustering the sequenced nucleic acid and identifying a candidate position of the unnatural base pair; determining a ratio of conversion of the intermediate to each one of a natural base pair at the candidate position of the unnatural base pair; comparing the ratio of conversion of the intermediate to a library of pre-determined conversion rate based on the sequences of one or more natural base pair adjacent to the candidate position of the unnatural base pair; wherein a substantial match of the ratio of conversion of the intermediate to a value in the library of the pre-determined conversion rate confirms the position of the unnatural base pair, thereby determining the sequence of the nucleic acid containing the unnatural base pair. Also disclosed is an apparatus for performing the method as disclosed herein.

### TECHNICAL FIELD

The present invention relates to nucleic acid chemistry. In particular, the invention relates to methods for sequencing nucleic acids that have an unnatural base pair.

### BACKGROUND

Watson-Crick base pairings, A-T and G-C, are among the most fundamental rules defining not only the central dogma of all living organisms on Earth but also current genetic engineering technology. However, this exclusive base pairing rule limits further advancements in biotechnology, because relying on only a four-letter genetic alphabet restricts the functionalities of nucleic acids and proteins. To overcome this limitation, genetic alphabet expansion of DNA by creating extra artificial base pairs (unnatural base pairs, UBPs) has attracted researchers' attention.

Recently, several types of UBPs that function as a third base pair in replication, transcription and/or translation have been created. Among them, Ds-Px (Ds: 7-(2-thienyl)-imidazo[4,5-b]pyridine and Px: diol-modified 2-nitro-4-propynylpyrrole) pair and P-Z pair have been subjected to an evolutionary engineering method, SELEX (Systematic Evolution of Ligands by EXponential enrichment), to generate unnatural base-containing DNA (UB-DNA) aptamers that specifically bind to target proteins and cells. The hydrophobic Ds bases in UB-DNA aptamers play an important role in augmenting the aptamers' affinities to targets. Semi-synthetic bacteria have also been created by incorporating a series of their UBPs, including 5SICS-NaM. The bacteria with the expanded genetic alphabet can produce proteins containing unnatural amino acids.

These advancements in genetic alphabet expansion technology are rapidly increasing the demands for a DNA sequencing method involving UBPs. In particular, the UB-DNA aptamer generation by SELEX requires a sequencing method that can determine the sequences of each aptamer candidate containing UBs in an enriched library, which is a mixture of different sequences obtained after several rounds of selection and amplification procedures in SELEX. Previously, a modified Sanger sequencing method was developed for a single DNA clone containing Ds bases. In the modified Sanger sequencing method, Ds positions appear as a gap over the natural base peak patterns. This sequencing method has been used for not only UB-DNA aptamer generation but also the creation of semi-synthetic bacteria to confirm the UB positions. However, to perform this sequencing method, each aptamer candidate clone must be isolated from the enriched library. In other words, to perform the sequencing method in the art, it is necessary to know the Ds positions in advanced. If the position of the Ds bases are not known, the sequencing method in the art would not be able to sequence the UBPs-containing DNAs. Therefore, there is a need to provide an alternative method of sequencing UBPs-containing DNAs.

### SUMMARY

In one aspect, there is provided a method of sequencing a nucleic acid containing an unnatural base pair (UBP), comprising performing two or more replacement replication reactions wherein the nucleic acid is replicated using two or more intermediate of the unnatural base pair; sequencing the nucleic acid resulting from the replacement replication reactions; clustering the sequenced nucleic acid and identifying a candidate position of the unnatural base pair; determining a ratio of conversion of the intermediate to each one of a natural base pair at the candidate position of the unnatural base pair; comparing the ratio of conversion of the intermediate to a library of pre-determined conversion rate based on the sequences of one or more natural base pair adjacent to the candidate position of the unnatural base pair; wherein a substantial match of the ratio of conversion of the intermediate to a value in the library of the pre-determined conversion rate confirms the position of the unnatural base pair, thereby determining the sequence of the nucleic acid containing the unnatural base pair.

In some examples, the method comprises two replacement replication reactions.

In some examples, the two replacement replication reactions comprise performing a first replacement replication reaction wherein the nucleic acid is replicated using a first intermediate of the unnatural base pair; and performing a second replacement replication reaction wherein the nucleic acid is replicated using a second intermediate of the unnatural base pair.

In some examples, the two replacement reactions are performed concurrently, sequentially, and/or separately.

In some examples, the first intermediate and the second intermediate are different intermediate of an unnatural base pair.

In some examples, the intermediate of the unnatural base pair is selected from the group consisting of Pa′, Pa, Pn, and Px.

In some examples, the unnatural base pair is composed of a nucleobase selected from the group consisting of:
a 7-(2-thienyl)imidazo[4,5-b]pyridin-3-yl group (Ds);
a 7-(2,2′-bithien-5-yl)imidazo[4,5-b]pyridin-3-yl group (Dss);
a 7-(2,2′,5′,2″-terthien-5-yl)imidazo[4,5-b]pyridin-3-yl group (Dsss);
a 2-amino-6-(2-thienyl)purin-9-yl group (s);
a 2-amino-6-(2,2′-bithien-5-yl)purin-9-yl group (ss);
a 2-amino-6-(2,2′,5′,2″-terthien-5-yl)purin-9-yl group (sss);
a 4-(2-thienyl)-pyrrolo[2,3-b]pyridin-1-yl group (dDsa);
a 4-(2,2′-bithien-5-yl)-pyrrolo[2,3-b]pyridin-1-yl group (Dsas);
a 4-[2-(2-thiazolyl)thien-5-yl]pyrrolo[2,3-b]pyridin-1-yl group (Dsav);
a 4-(2-thiazolyl)-pyrrolo[2,3-b]pyridin-1-yl group (dDva);
a 4-[5-(2-thienyl)thiazol-2-yl]pyrrolo[2,3-b]pyridin-1-yl group (Dvas);
a 4-(2-imidazolyl)-pyrrolo[2,3-b]pyridin-1-yl group (dDia); and

a Ds derivative:

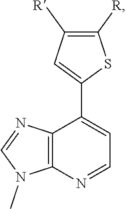

wherein R and R′ each independently represent any moiety represented by the following formula:

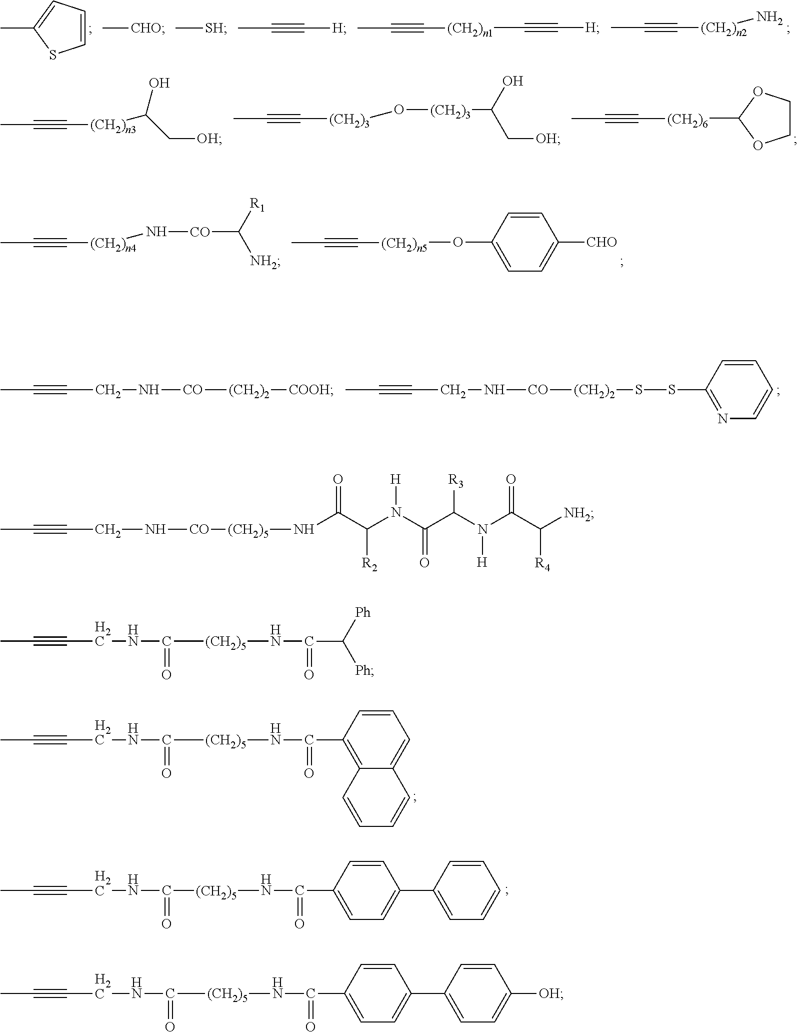

wherein n1=2 to 10; n2=1 or 3; n3=1, 6, or 9; n4=1 or 3; n5=3 or 6; R1=Phe (phenylalanine), Tyr (tyrosine), Trp (tryptophan), His (histidine), Ser (serine), or Lys (lysine); and R2, R3, and R4=Leu (leucine), Leu, and Leu, respectively, or Trp, Phe, and Pro (proline), respectively.

In some examples, the natural base pair is composed of a nucleobase selected from the group consisting of A, G, C, U, and T.

In some examples, the nucleic acid is a DNA strand.

In some examples, the library of pre-determined conversion rate comprises a ratio of the conversion of an unnatural base pair to either one of a natural base pair.

In some examples, the library of pre-determined conversion rate comprises a ratio of the conversion of an unnatural base pair to either one of a natural base pair based on the sequence of one or more adjacent base pair.

In some examples, the replacement replication reaction further comprises replicating the nucleic acid using natural base pairs.

In some examples, the replacement replication reaction is a replacement polymerase chain reaction (PCR).

In some examples, the replacement replication reaction comprises

performing a first nucleic acid replication reaction using a first replication substrate containing an intermediate of the unnatural base pair to thereby replace the unnatural base pair with the intermediate of the unnatural base pair; and

performing a second nucleic acid replication reaction using a second replication substrate containing natural base pair to thereby replace the intermediate of the unnatural base pair with a natural base pair.

In some examples, the replacement replication reaction further comprises

replicating or amplification of the nucleic acid from the second nucleic acid replication reaction to thereby have a plurality of nucleic acid with natural base pair resulting from the second nucleic acid replication reaction.

In some examples, the sequencing is performed using deep sequencing method.

In some examples, the identifying the candidate position of the unnatural base pair comprises aligning the sequenced nucleic acid and determining a position that contains varying nucleobase.

In some examples, the ratio of conversion of the intermediate to each one of a natural base pair at the candidate position of the unnatural base pair is calculated using the formula:

% rA (at position i)=CR(A,i)=S(A,i)/[S(A,i)+S(G,i)+S(C,i)+S(T,i)]×100

where S(n, i) is the read numbers of sequences which has natural base n at position i.

In some examples, the substantial match of the ratio of conversion of the intermediate is a value that is within about 10% of the value in the library of the pre-determined conversion rate.

In another aspect, there is provided an apparatus for performing the method of any one of the preceding claims.

### BRIEF DESCRIPTION OF THE DRAWINGS

Exemplary embodiments of the invention will be better understood and readily apparent to one of ordinary skill in the art from the following written description, by way of example only, and in conjunction with the drawings, in which:

FIG. 1 is an exemplary workflow of the present disclosure. FIG. 1(A) shows the chemical structures of the natural A-T and G-C pairs, the unnatural Ds-Px pair and the unnatural Px derivative bases, Pa, Pa′, and Pn. FIG. 1(B) shows the sequencing scheme for Ds-containing DNA. The Ds base in the sequence is replaced with the natural bases, mainly A or T, through short cycles of replacement PCR in the presence of the natural dNTPs and the additional unnatural Pa′ or other unnatural base substrates (such as Pa, Pn, or Px), before conventional deep sequencing. The resultant natural-base composition rates will differ, depending on the replacement PCR process.

FIG. 2 shows a schematic diagram of the concept for generating an encyclopedia from the data obtained by deep sequencing of the replacement PCR products using authentic Ds-containing libraries. Natural-base composition rates will differ, depending on the local sequence context surrounding the Ds bases.

FIG. 3 shows an exemplary analysis of replacement PCR using an intermediate UB substrate, Pa′, reduces the sequence bias in the contexts surrounding the Ds base. FIG. 3(A) is a scheme of the Ds replacement with natural bases without/with the Pa′ substrate in replacement PCR. FIG. 3(B-C) are heat maps indicating natural-base-replacement efficiencies without (B) or with the Pa′ substrate (C) for each sequence context surrounding the Ds base. Read counts were normalized to reads per million (RPM).

FIG. 4 shows examples of the compositions of the replaced natural bases and the replacement efficiencies, which depend on the local sequence contexts surrounding the Ds base. Representative examples of replaced natural bases and the efficiencies for the six different replacement PCR conditions investigated in this study. Among the whole sequence data in each replacement PCR condition (FIG. 8-13), some sequence contexts were chosen. They were categorized into four groups based on the read count distribution, Ds→A rate, Ds→T rate and Ds→G/C rate. Each color represents the natural base replaced from the Ds base (solid, A; dotted, T; lined, G; open, C).

FIG. 5 shows a schematic diagram of an exemplary process of determining the sequences of Ds-containing DNAs. The Ds base in the sequence is replaced through two replacement PCR methods, in the presence of either dPa′TP or dPxTP, and their sequence data are obtained by deep sequencing. Natural-base composition rates depend on the local sequence context surrounding the Ds base. Thus, the A/T ratios at A/T variable sites in a clustered sequence family are scanned using a prepared “Encyclopedia” (ENBRE), composed of the training data of the natural base replacement patterns for 4⁶ local sequence contexts. The replacement patterns also depend on the replacement PCR conditions, and thus a position with varying A/T ratios depending on each condition, and with ratios that are close to the reference values in the encyclopedia, can be identified as a possible Ds position.

FIG. 6 refers to the encyclopedia data allows for simple and fast determination of the Ds positions. FIG. 6(A) shows an experimental scheme for sequencing Ds-containing DNA libraries for UB-DNA aptamer generation. FIG. 6(B-C) shows alignments of family 1 anti-IFNγ aptamer clones determined by deep sequencing analyses. The natural-base composition rates at each position are shown in FIG. 17. The most frequent sequence in family 1 is shown in the top row and the variations in the bases are coloured (solid, A; dotted, T; greyed, G; open, C). Three Ds bases at predetermined positions (shown by arrows) were replaced with natural bases in the replacement PCR with dPa′TP (B) or with dPxTP (C). The proportion of each sequence appearing in the deep sequencing is indicated in the first column. Among the biological triplicate data, one set is shown as the representative. FIG. 6(D) shows a comparison of the Ds→A conversion rate (% rA) between the ENBRE data and the actual sequence data for the three Ds positions in the family 1 anti-IFNγ aptamer sequence. The % rA values in the obtained sequence data were calculated as an average in the biological experiments, performed in triplicate. FIG. 6(E) shows a schematic illustration of the secondary structure of the anti-IFNγ UB-DNA aptamer as known in the art.

FIG. 7 shows a comparison of the replacement patterns between two conditions enables the Ds positions to be distinguished from other natural-base positions. FIG. 7(A-B) Alignment of the top families, obtained from the enriched library #1 (A) and library #4 (B) for anti-vWF aptamer generation, after replacement PCR using dPa′TP. Three or two Ds bases at the positions indicated with red arrows were replaced with natural bases. The natural-base composition rates at each position are shown in FIG. 17B. Among the duplicated data analyses, one set is shown as the representative. FIG. 7(C) Comparison of the Ds→A conversion rate (% rA) between the ENBRE data and the actual sequence data for three Ds positions. The % rA values in the actual sequence data were calculated as an average in the technical sequencing, which was performed in duplicate. FIG. 7(D) Schematic illustration of the secondary structure of the anti-vWF UB DNA aptamer. This aptamer was obtained from two enriched selection libraries, #1 and #4. The sequence difference between the two was Ds or T at position 22, which was confirmed in a previous sequencing method based on the Sanger approach.

FIG. 8 shows the natural base replacement efficiencies for each sequence context of NDsN2-29 in cond. 1 (UB−/Accuprime Pfx DNA pol). Each bar plot shows read counts for each sequence context determined by deep sequencing analyses after replacement PCR of DsN2-49. Read counts were normalised to reads per million (RPM). Each color represents the natural base replaced with the Ds base (solid, A; dotted, T; lined, G; open, C).

FIG. 9 shows the natural base replacement efficiencies for each sequence context of NDsN2-49 in cond. 2 (Pa′+/AccuPrime Pfx DNA pol). Each color represents the natural base replaced with the Ds base (solid, A; dotted, T; lined, G; open, C).

FIG. 10 shows the natural base replacement efficiencies for each sequence context NDsN2-49 in cond. 3 (Pa+/AccuPrime Pfx DNA pol). Each color represents the natural base replaced with the Ds base (solid, A; dotted, T; lined, G; open, C).

FIG. 11 shows the natural base replacement efficiencies for each sequence context of NDsN2-49 in cond. 4 (Px+/AccuPrime Pfx DNA pol). Each color represents the natural base replaced with the Ds base (solid, A; dotted, T; lined, G; open, C).

FIG. 12 shows the natural base replacement efficiencies for each sequence context of NDsN2-49 in cond. 5 (UB−/Taq DNA pol). Each color represents the natural base replaced with the Ds base (solid, A; dotted, T; lined, G; open, C).

FIG. 13 shows the natural base replacement efficiencies for each sequence context of NDsN2-49 in cond. 6 (Pa′+/Taq DNA pol). Each color represents the natural base replaced with the Ds base (solid, A; dotted, T; lined, G; open, C).

FIG. 14 shows the low natural base replacement biases in replacement PCR by using Pa′ or Px with AccuPrime Pfx DNA pol. FIG. 14(A) shows the relative read counts based on extracted sequence lengths under each replacement PCR conditions (cond.1 to cond.6). The y-axis represents the ratio of reads of each length and 100% represents the total read counts of 1 to 20 bases surrounded by primer annealing regions (see Materials and Methods). FIG. 14(B) shows the histogram of read counts for 256 sequence contexts determined by deep sequencing analyses after replacement PCR of NDsN2-49 under six different conditions.

FIG. 15 shows boxplots showing the percentage of each natural base replaced from the Ds base (% rN, natural-base composition rate) in 256 sequence contexts of NDsN2-49. Each panel plots data obtained from replacement PCR under different conditions. Triangles represent the mean.

FIG. 16 shows scatter plots showing the reproducibility of the Ds conversion rate for 4,096 sequence contexts of NDsN3-49. The average and standard deviation (consistency) of the Ds→A rate (% rA, shown in A) and Ds→T rate (% rT, shown in B) in biological triplicates were calculated for each replacement PCR with dPa′TP or dPxTP.

FIG. 17 shows the comparison of natural-base composition rates at each base with ENBRE. Conversion rates to each natural base (% rN) in the top-ranked clustered sequences (family 1) were calculated, by using sequence reads obtained from replacement PCR with either dPa′TP or dPxTP of each enriched library. The rates were compared with those in ENBRE. FIG. 17(A) shows N43Ds-P001 mix (anti-IFNγ UB-DNA aptamer). FIG. 17(B) shows N30Ds-S6-006 libraries #1 and #4 (anti-vWF UB-DNA aptamer).

FIG. 18 shows the accuracy, sensitivity and specificity for determining the Ds positions using ENBRE. FIG. 18(A) shows an example of the initial scanning for the Ds positions. For example, at all A positions in the family 1 anti-IFNγ aptamer sequence (top-ranked), the % rA values were compared with the corresponding reference % rA values in ENBRE, assuming that the Ds base is located in each sequence context. A positive value means that the reference value in ENBRE was higher than the actual value. FIG. 18(B) shows the accuracy of ENBRE to predict % rA values. The y-axis represents the % rA deviation [Error %=(reference value in ENBRE)−(% rA obtained from actual sequence data)]. In the two replacement PCR methods using dPa′TP or dPxTP, the calculated deviations for the total of 20 original Ds positions in the top ten family anti-IFNγ aptamer sequences were plotted. Triangles represent the mean. FIG. 18(C) shows a flow chart for determining the Ds positions using ENBRE. FIG. 18(D) shows the ROC curve analysis of the case of the anti-IFNγ aptamer selection (see Materials and Methods). The sensitivity (true positive rate) and the specificity (1—false positive rate) are shown in the table when the acceptable error range for criterion 1 was ±10% (shown in black dots). Even if % rA does not match well with ENBRE, the use of criterion 2 increases the sensitivity without a loss of specificity (shown in solid lines).

### DETAILED DESCRIPTION

The creation of unnatural base pairs (UBPs) has rapidly advanced the genetic alphabet expansion technology of DNA, requiring a new sequencing method for UB-containing DNAs with five or more letters. The hydrophobic UBP, Ds-Px, exhibits high fidelity in PCR and has been applied to DNA aptamer generation involving Ds as a fifth base. The present disclosure describes a sequencing method for UBP (such as Ds-Px)-containing DNAs, in which the UBP (such as Ds-Px) bases are replaced with natural bases by PCR using intermediate UB substrates (replacement PCR) for conventional deep sequencing. The inventors of the present disclosure found that the composition rates (i.e. conversion rates) of the natural bases converted from the UBs (such as Ds) significantly varied (or is unique) depending on the sequence contexts around the UB (such as Ds) and one or more different intermediate substrates. Using the finding that the composition rate or conversion rate of natural bases converted from UBs (such as Ds) varies (or is unique) to the sequence context around the UB, the inventors of the present disclosure developed an encyclopedia (or library) of the natural-base composition (or conversion) rates corresponding to all of the sequence contexts for each replacement PCR method using different intermediate substrates. The inventors found that using the encyclopedia/library, the UBPs positions in DNAs can be determined by comparing the natural-base composition/conversion rates in both the actual and encyclopedia data (i.e. library data), at each position of the DNAs obtained by deep sequencing after replacement PCR.

Therefore, in one aspect, there is provided a method of sequencing a nucleic acid containing an unnatural base pair (UBP), comprising performing two or more replacement replication reactions wherein the nucleic acid is replicated using two or more intermediate of the unnatural base pair; sequencing the nucleic acid resulting from the replacement replication reactions; clustering the sequenced nucleic acid and identifying a candidate position of the unnatural base pair; determining a ratio of conversion of the intermediate to each one of a natural base pair at the candidate position of the unnatural base pair; comparing the ratio of conversion of the intermediate to a library of pre-determined conversion/composition rate based on the sequences of one or more natural base pair adjacent to the candidate position of the unnatural base pair; wherein a substantial match of the ratio of conversion of the intermediate to a value in the library of the pre-determined conversion/composition rate confirms the position of the unnatural base pair, thereby determining the sequence of the nucleic acid containing the unnatural base pair.

In some examples, wherein the method further comprises a second replacement replication reaction wherein the nucleic acid is replicated using a second intermediate of the unnatural base pair. In some examples, the method may comprise two replacement replication reactions. In such examples, the two replacement replication reactions may comprise performing a first replacement replication reaction wherein the nucleic acid is replicated using a first intermediate of the unnatural base pair; and performing a second replacement replication reaction wherein the nucleic acid is replicated using a second intermediate of the unnatural base pair. As such, in some examples, the two replacement reactions may be performed concurrently, sequentially, and/or separately.

In some examples, the method of sequencing a nucleic acid containing an unnatural base pair (UBP) of the present disclosure may comprise performing a first replacement replication reaction wherein the nucleic acid is replicated using a first intermediate of the unnatural nucleobase; performing a second replacement replication reaction wherein the nucleic acid is replicated using a second intermediate of the unnatural nucleobase; sequencing the nucleic acid resulting from the first and second replacement replication reactions; clustering the sequenced nucleic acid and identifying a candidate position of the unnatural nucleobase; determining a first ratio of conversion of the first intermediate to each nucleobase of a natural nucleobase at the candidate position of the unnatural nucleobase; determining a second ratio of conversion of the second intermediate to each nucleobase of a natural nucleobase at the candidate position of the unnatural nucleobase; comparing the first ratio and the second ratio to a library of pre-determined composition rate based on the sequences of the natural nucleobases adjacent to the candidate position of the unnatural nucleobase; wherein a substantial match of the first ratio and the second ratio to the pre-determined composition rate confirms the position of the unnatural base pair, thereby determining the sequence of the nucleic acid containing the unnatural base pair.

In some examples, the present disclosure also provides a method of identifying the position of an unnatural base pair (UBP) in a nucleic acid sequence, comprising the steps as described above. For example, the method may comprise performing a first replacement replication reaction wherein the nucleic acid is replicated on a first template comprising a first intermediate of the unnatural base pair; performing a second replacement replication reaction wherein the nucleic acid is replicated on a second template comprising a second intermediate of the unnatural base pair; sequencing the nucleic acid resulting from the first and second replacement replication reactions; clustering the sequenced nucleic acid and identifying a candidate position of the unnatural base pair; determining a first ratio of conversion of the first intermediate to each base of a natural base pair at the candidate position of the unnatural base pair; determining a second ratio of conversion of the second intermediate to each base of a natural base pair at the candidate position of the unnatural base pair; comparing the first ratio and the second ratio to a library of pre-determined composition rate based on the sequences of the natural base pair adjacent to the candidate position of the unnatural base pair; wherein a substantial match of the first ratio and the second ratio to the pre-determined composition rate confirms the position of the unnatural base pair, thereby identifying the position of the unnatural base pair.

Conversely, the method as described herein may comprise three, or four, or five or more replacement replication reactions wherein the nucleic acid is replicated using a third intermediate, or a fourth intermediate, or fifth intermediate, or more intermediate of the unnatural base pair.

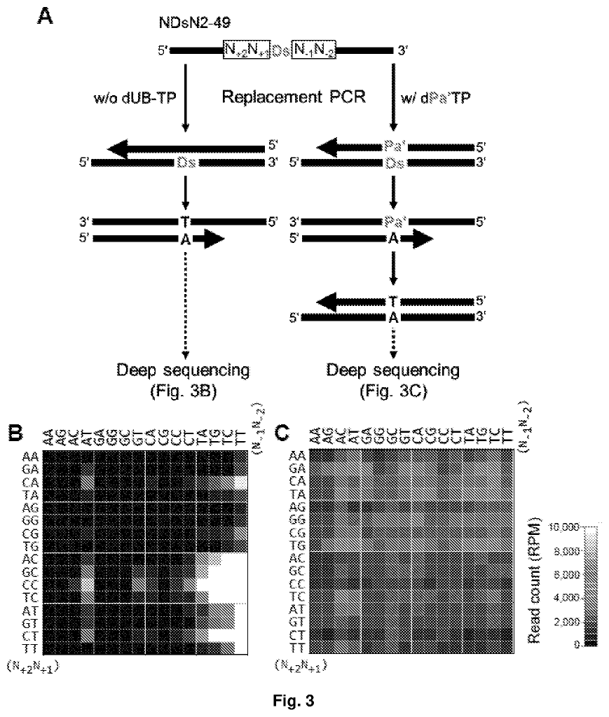

The use of the intermediate substrate of the unnatural base pair was found to be useful by the inventors of the present disclosure. For example, when replacement PCR is performed without an intermediate substrate of the unnatural base pair, the replacement PCR was found to have greatly reduced conversion efficiency (see FIG. 3A left column and FIG. 3B for the resulting conversion).

To provide an additional parameter that can be utilized to determine the sequence of a nucleic acid containing an unnatural base pair, in some examples, the one or more intermediate may be different intermediate of the same unnatural base pair. For example, the first intermediate and the second intermediate are different intermediate of an unnatural base pair. In some examples, where the unnatural base pair is composed of an unnatural base 7-(2-thienyl)imidazo[4,5-b]pyridin-3-yl group (i.e. Ds), the intermediate of the unnatural base may include, but is not limited to, Pa′, Pa, Pn, Px, and the like. The intermediate of are as follows:

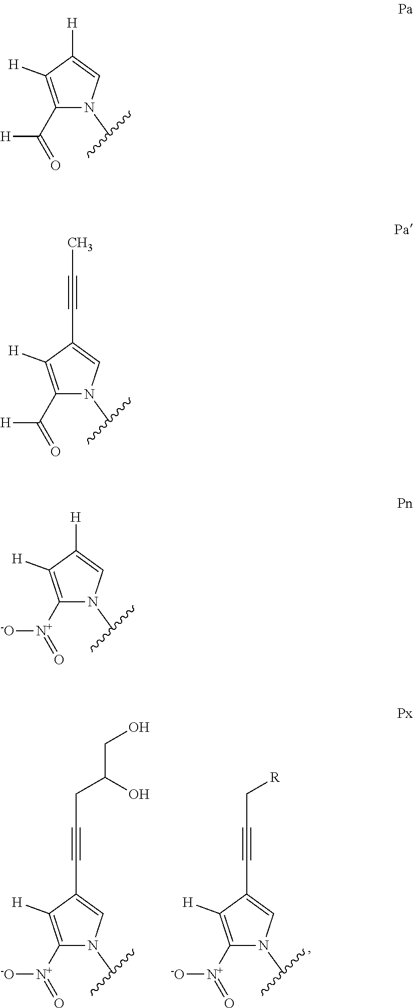

wherein R may be any one of the following functional groups:

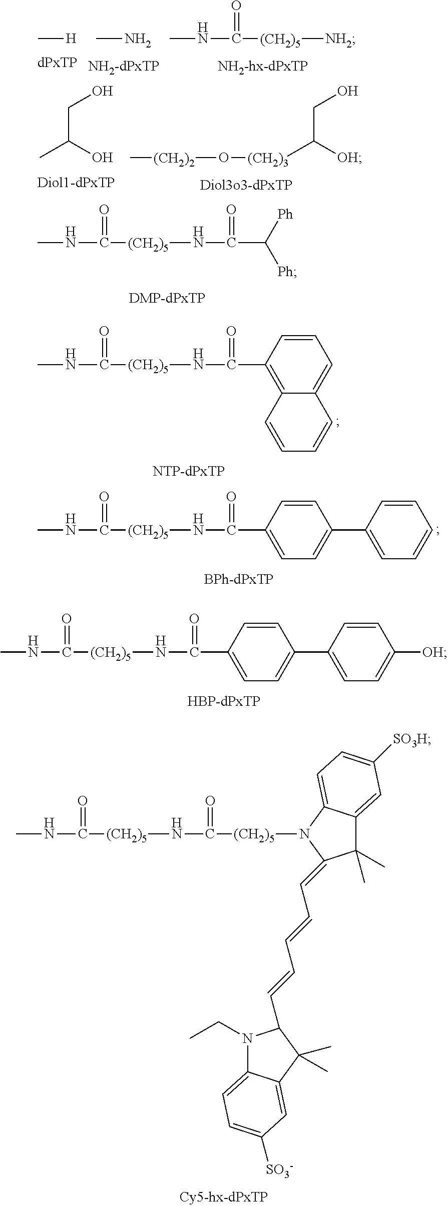

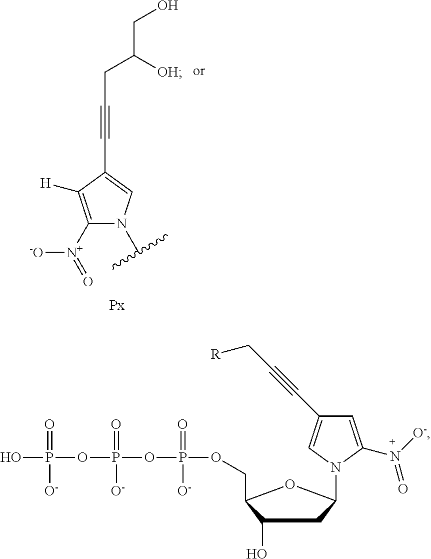

where R may be any one of:

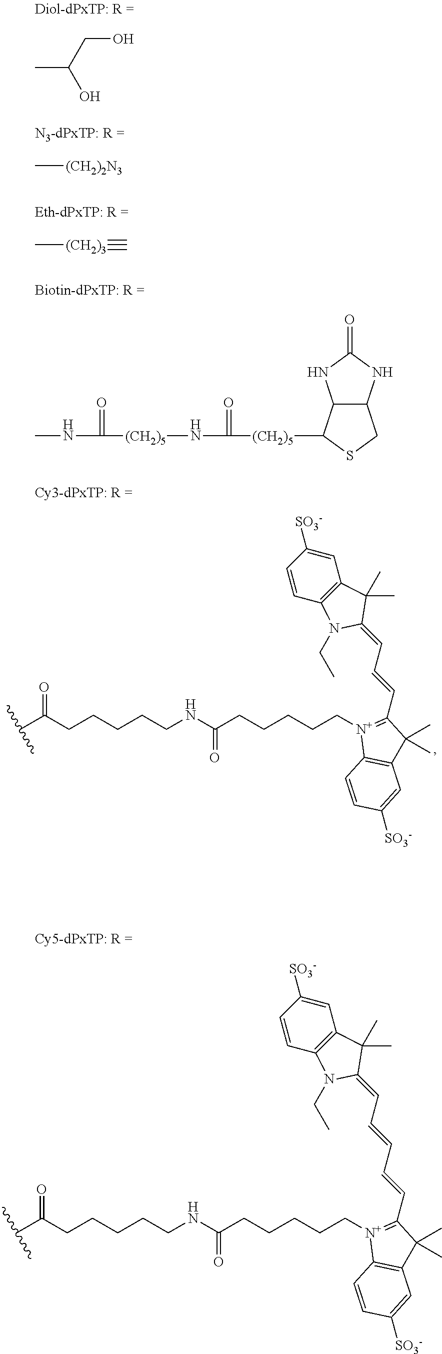

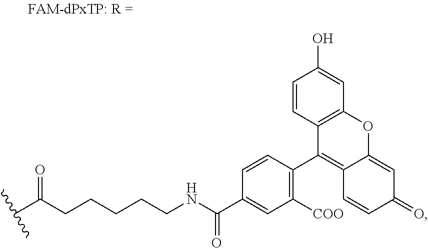

or

a Pn derivatives, such as:

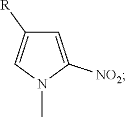

where R represents any moiety represented by the following formula:

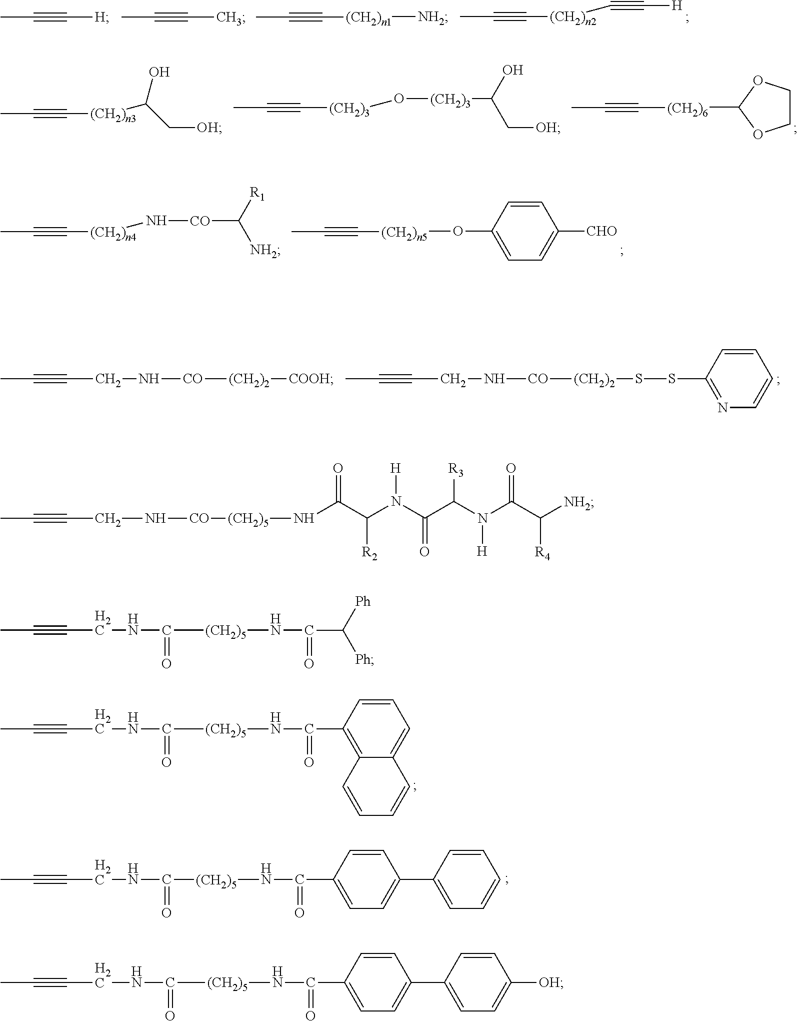

wherein n1=1 or 3, n2=2 to 10, n3=1, 6, 9; n4=1 or 2, n5=3 or 6; R1=Phe, Tyr, Trp, His, Ser, or Lys; and R2, R3, and R4=Leu, Leu, and Leu, respectively, or Trp, Phe, and Pro, respectively; or

a Pa derivative such as

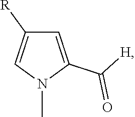

wherein R represents any moiety represented by the following formula:

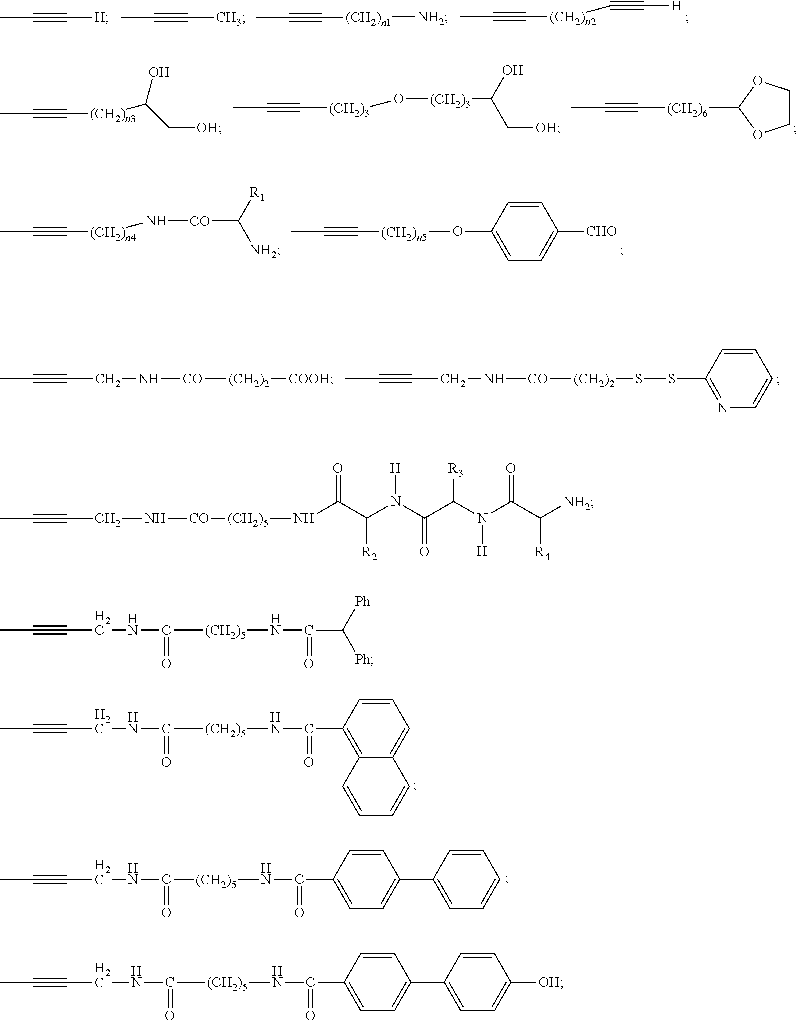

wherein n1=1 or 3; n2=2 to 10; n3=1, 6, or 9; n4=1 or 3; n5=3 or 6; R1=Phe, Tyr, Trp, His, Ser, or Lys; and R2, R3, and R4=Leu, Leu, and Leu, respectively, or Trp, Phe, and Pro, respectively.

As would be appreciated by the person skilled in the art, Pn is R═H (no propynyl group/triple bond), 2-nitropyrrole; and wherein, Px is used for the derivatives with the triple bond.

In some examples, the intermediate may be provided as substrates suitable for replacement replication reaction (for example replacement PCR). In some examples, the intermediate may be a triphosphate substrate of an unnatural base pair. In some examples, the intermediate may be provided as substrates such as, but is not limited to, dPa′TP, dPaTP, dPnTP and/or dPxTP. In some examples, the first intermediate and the second intermediate are not the same intermediate of the unnatural base pair. In some examples, one of the first or second intermediate may be dPa′TP. In some examples, one of the first or second intermediate may be dPxTP. When the first intermediate is dPa′TP, the second intermediate will be dPxTP, and vice versa.

As used herein, the term “unnatural base pair” refers to a nucleic acid base pair composed of artificially made or non-standard pair of nucleobases. Thus, in some examples, the unnatural base pair is composed of a nucleobase (or an unnatural base) such as, but is not limited to:
a 7-(2-thienyl)imidazo[4,5-b]pyridin-3-yl group (Ds);
a 7-(2,2′-bithien-5-yl)imidazo[4,5-b]pyridin-3-yl group (Dss);
a 7-(2,2′,5′,2″-terthien-5-yl)imidazo[4,5-b]pyridin-3-yl group (Dsss);
a 2-amino-6-(2-thienyl)purin-9-yl group (s);
a 2-amino-6-(2,2′-bithien-5-yl)purin-9-yl group (ss);
a 2-amino-6-(2,2′,5′,2″-terthien-5-yl)purin-9-yl group (sss);
a 4-(2-thienyl)-pyrrolo[2,3-b]pyridin-1-yl group (dDsa);
a 4-(2,2′-bithien-5-yl)-pyrrolo[2,3-b]pyridin-1-yl group (Dsas);
a 4-[2-(2-thiazolyl)thien-5-yl]pyrrolo[2,3-b]pyridin-1-yl group (Dsav);
a 4-(2-thiazolyl)-pyrrolo[2,3-b]pyridin-1-yl group (dDva);
a 4-[5-(2-thienyl)thiazol-2-yl]pyrrolo[2,3-b]pyridin-1-yl group (Dvas);
a 4-(2-imidazolyl)-pyrrolo[2,3-b]pyridin-1-yl group (dDia); or

a Ds derivatives, such as:

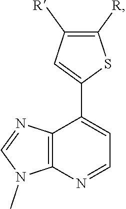

wherein R and R′ each independently represent any moiety represented by the following formula:

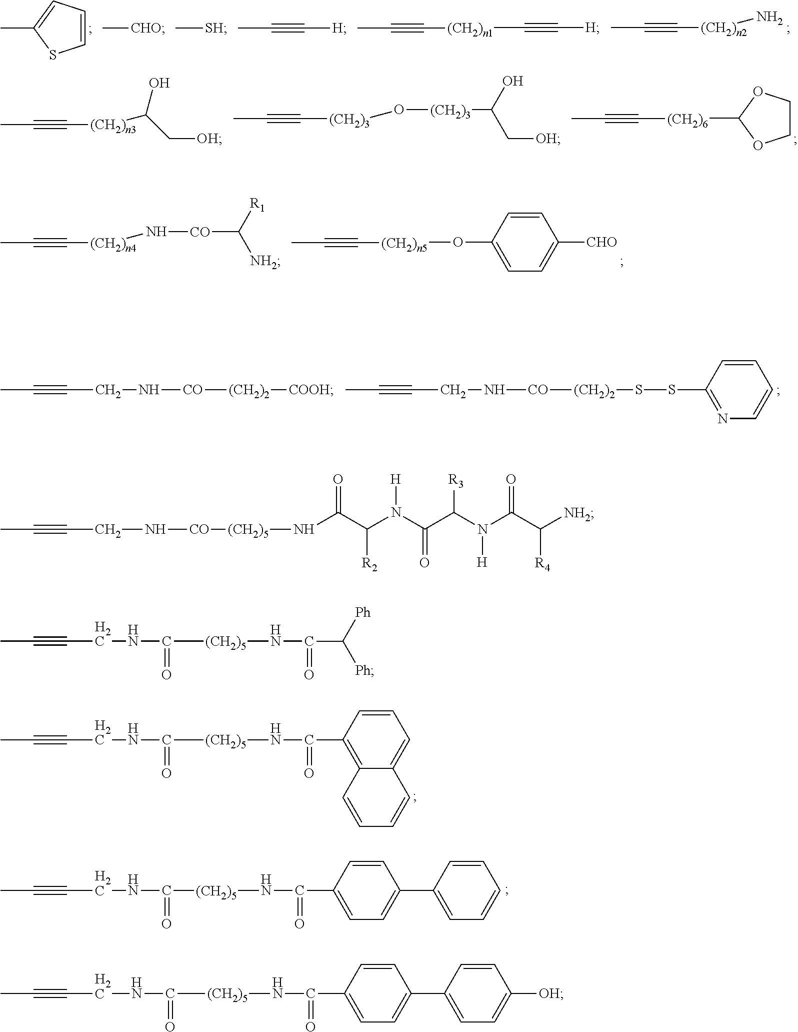

wherein n1=2 to 10; n2=1 or 3; n3=1, 6, or 9; n4=1 or 3; n5=3 or 6; R1=Phe (phenylalanine), Tyr (tyrosine), Trp (tryptophan), His (histidine), Ser (serine), or Lys (lysine); and R2, R3, and R4=Leu (leucine), Leu, and Leu, respectively, or Trp, Phe, and Pro (proline), respectively.

However, it would be understood by the person skilled in the art that the method as described herein may be used on any unnatural base pairs known in the art, provided the intermediate of the unnatural base pairs is known.

In some example, the unnatural base pair may be a Ds-Px pair as follows:

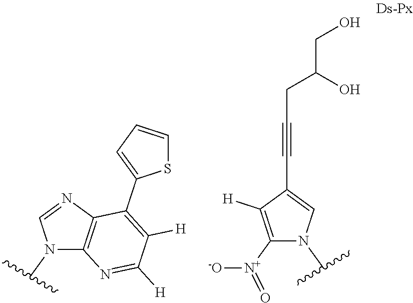

In contrast to the term “unnatural base pair”, as used herein, the term “natural base pair” that refers to a nucleic acid base composed of standard or naturally occurring pair of nucleobases such as adenine (A), guanine (G), thymine (T), uracil (U), and cytosine (C). Thus, in some examples, the natural base pair may be composed of a nucleobase selected from the group consisting of A, G, C, U, and T.

In some examples, the nucleic acid as described herein includes nucleic acid sequences that comprises one or more natural base pair and one or more unnatural base pair. In some examples, the nucleic acid described herein includes nucleic acids with no more than 20% unnatural base pairs, or no more than 15% unnatural base pairs, or no more than 14% unnatural base pairs, or no more than 13% unnatural base pairs, or no more than 12% unnatural base pairs, or no more than 11% unnatural base pairs, or no more than 10% unnatural base pairs, or no more than 9% unnatural base pairs, or no more than 8% unnatural base pairs, or no more than 7% unnatural base pairs, or no more than 6% unnatural base pairs, or no more than 5% unnatural base pairs, or no more than 4% unnatural base pairs, or no more than 3% unnatural base pairs, or no more than 2% unnatural base pairs, or no more than 1% unnatural base pairs.

In some examples, the nucleic acid having a template of 5′-N₊₂N₊₁X_{Y}N₋₁N₋₂-3′ may include no more than 20% unnatural base pairs, or no more than 15% unnatural base pairs, or no more than 14% unnatural base pairs, or no more than 13% unnatural base pairs, or no more than 12% unnatural base pairs, or no more than 11% unnatural base pairs, or no more than 10% unnatural base pairs, or no more than 9% unnatural base pairs, or no more than 8% unnatural base pairs, or no more than 7% unnatural base pairs, or no more than 6% unnatural base pairs, or no more than 5% unnatural base pairs, or no more than 4% unnatural base pairs, or no more than 3% unnatural base pairs, or no more than 2% unnatural base pairs, or no more than 1% unnatural base pairs.

In some examples, the nucleic acid having a template of 5′-N₊₃N₊₂N₊₁X_{Y}N₋₁N₋₂N₋₃-3′ may include no more than 15% unnatural base pairs, or no more than 14% unnatural base pairs, or no more than 13% unnatural base pairs, or no more than 12% unnatural base pairs, or no more than 11% unnatural base pairs, or no more than 10% unnatural base pairs, or no more than 9% unnatural base pairs, or no more than 8% unnatural base pairs, or no more than 7% unnatural base pairs, or no more than 6% unnatural base pairs, or no more than 5% unnatural base pairs, or no more than 4% unnatural base pairs, or no more than 3% unnatural base pairs, or no more than 2% unnatural base pairs, or no more than 1% unnatural base pairs.

It is believed that the method as presently disclosed may be used for the sequencing of either DNA and/or RNA strand. Thus, the method of the present disclosure may be performed on nucleic acid that is a DNA and/or RNA strand. In some examples, the nucleic acid may be a DNA and/or RNA strand. In some examples, the nucleic acid is a DNA strand. When the nucleic acid is a DNA strand, the natural base pair is composed of natural nucleobases such as A, G, C, and T. In some examples, the natural base pair may be as follows:

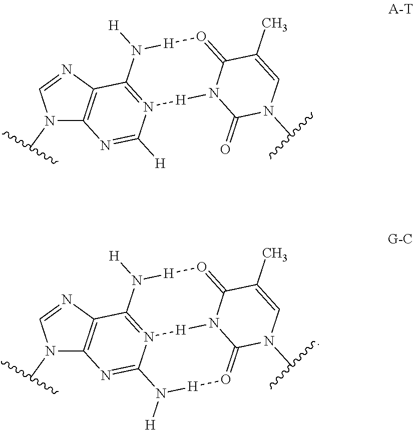

The inventors of the present disclosure found that the ratio of the conversion/composition of an unnatural base pair to either one of a natural base pair varies (and is unique) depending on the sequence of the natural base pair immediately adjacent to the position of the unnatural base pair. Thus, the variation and the uniqueness of the ratio of the conversion can be used as a reference when determining the presence or absence of an unnatural base pair.

As used herein, the term “composition rate” or “conversion rate” may be used interchangeably to refer to the probability (or rate) of an unnatural base pair being replaced (in a replacement PCR) by one of four natural nucleobases in context (or depending on) the sequence of the one or more natural nucleobase immediately adjacent to the position of the unnatural base pair.

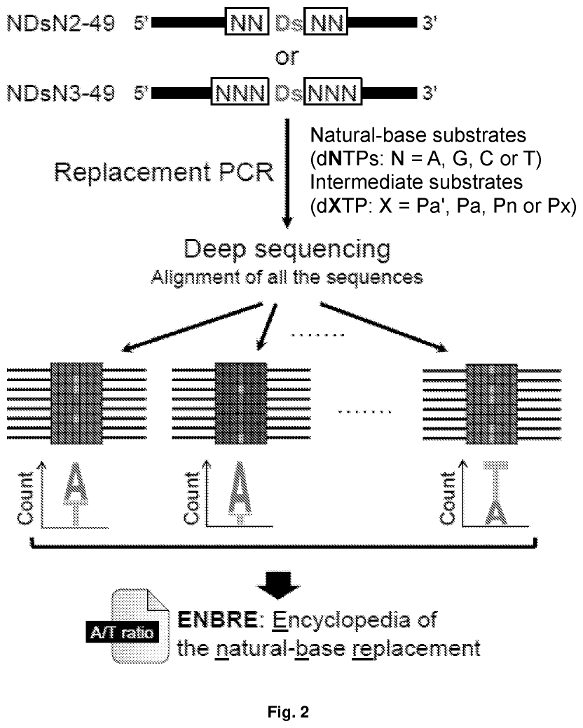

As exemplified in the Experimental section below and in FIG. 2, the library of pre-determined conversion/composition rate may be generated using a DNA library containing natural nucleobase (i.e. natural-base) randomized sequences and an unnatural base pair (such as a Ds). In some examples, the library of pre-determined conversion/composition rate comprises a ratio of the conversion of an unnatural base pair to either one of a natural base pair. One possible example of the library of pre-determined conversion/composition rate is Table 3. However, it would be generally understood that such library would be readily generated using the concept as described in the present disclosure.

In some examples, the library of pre-determined conversion/composition rate may be generated by (1) providing a plurality of template nucleic acid containing natural nucleobase (i.e. natural-base) randomized sequences and an unnatural base pair (such as a Ds); (2) performing a replacement replication reaction on the plurality of template nucleic acid with one intermediate of the unnatural base pair (or nucleobase); (3) performing further replacement replication reaction on the nucleic acid from (2) with natural base pair (or nucleobase) to thereby have a plurality of nucleic acid with no unnatural base pair (or nucleobase); (4) sequencing the resulting nucleic acid from (3); (5) clustering the sequences of the nucleic acid obtained from the sequencing step and/or identifying the position of the unnatural base pair (or nucleobase); (6) determining a ratio (or rate or probability) of conversion of the unnatural base pair (or nucleobase) to each of the natural base pair (or nucleobase); wherein the ratio is a value point (data point) in the library of pre-determined conversion/composition rate that is unique to the sequence of the template nucleic acid. The value point/ratio/rate/data point in the library of each template nucleic acid sequence serves as a unique identification point of the nucleic acid sequence that contains the unnatural base pair (or nucleobase). In order to build the library, it would be advantages if the sequence of the plurality of the template nucleic acid in (1) is known or pre-determined or pre-designed. In some examples, the plurality of template nucleic acid may be in the format of 5′-N₊₁X_{Y}N₋₁-3′, 5′-N₊₂N₊₁X_{Y}N₋₁N₋₂-3′, 5′-N₊₃N₊₂N₊₁X_{Y}N₋₁N₋₂N₋₃-3′, 5′-N_{+M}N_{+(M-1) . . .} N₊₂N₊₁X_{Y}N₋₁N_{-2 . . .} N_{-(M-1)}N_{-M}-3′, and the like, wherein X is an unnatural nucleobase (for example a Ds), N is independently any one of A, G, C, or U/T, Y is an integer having a value of 1 to 3, and M is an integer having a value of 1 to 50. In some examples, M may be 30, 31, 32, 33, 34, 35, 36, 37, 38, 39, or 40.

Thus, the library of pre-determined conversion/composition rates includes the conversion rate of an unnatural base pair to either one of a natural base pair based on the sequence of one or more natural base pair immediately adjacent to the position of the unnatural base pair. In some examples, the library of pre-determined conversion/composition rate comprises a ratio of the conversion of an unnatural base pair to either one of a natural base pair based on the sequence of one, or two, or three, or four, or five, or six, or seven, or eight, or nine, or ten natural base pair (immediately) adjacent to the unnatural base pair. In some examples, the library of pre-determined conversion/composition rates may include the conversion rate of 5′-N₊₁X_{Y}N₋₁-3′, the conversion rate of 5′-N₊₂N₊₁X_{Y}N₋₁N₋₂-3′, the conversion rate of 5′-N₊₃N₊₂N₊₁X_{Y}N₋₁N₋₂N₋₃- 3′, the conversion rate of 5′-N_{+M}N_{+(M-1) . . .} N₊₂N₊₁X_{Y}N₋₁N_{-2 . . .} N_{-(M-1)}N_{-M}-3′, and the like, wherein X is an unnatural nucleobase (for example a Ds), N is independently any one of A, G, C, or U/T, Y is an integer having a value of 1 to 3, and M is an integer having a value of 1 to 50. In some examples, M may be 30, 31, 32, 33, 34, 35, 36, 37, 38, 39, or 40.

In some examples, the library of pre-determined composition rate comprises a ratio or the probability of the conversion of an unnatural nucleobase to either one of a natural nucleobase depending on the sequence of one or more adjacent nucleobase. In some examples, the composition rate may be calculated using the following formula:

$$C R ( n , i ) = \frac{S ( n , i )}{{\sum }_{X = A , C , G , T} S ( n , X )} \times 100$$

where S(n, i) is the read numbers of sequences which has natural base n at position i, and CR(n, i) is the composition rate to natural base n at position i.

In some examples, the composition rate may be calculated using the formula: CR (n, i)=% rN (at position i)=S(n, i)/[S(A, i)+S(G, i)+S(C, i)+S(T, i)]×100, where S(n, i) is the read numbers of sequences which has natural base n at position i, and CR(n, i) is the composition rate to natural base n at position i.

In some examples, the replacement replication reaction further comprises replicating the nucleic acid using natural base pairs.

In some examples, the replacement replication reaction may be a replacement polymerase chain reaction (PCR). In some examples, where the nucleic acid is an RNA strand, the replacement replication reaction may include a reverse transcription followed by a replacement polymerase chain reaction (PCR). In some examples, where the nucleic acid is a strand of RNA, reverse transcription may be included, and primer extension may also be utilised.

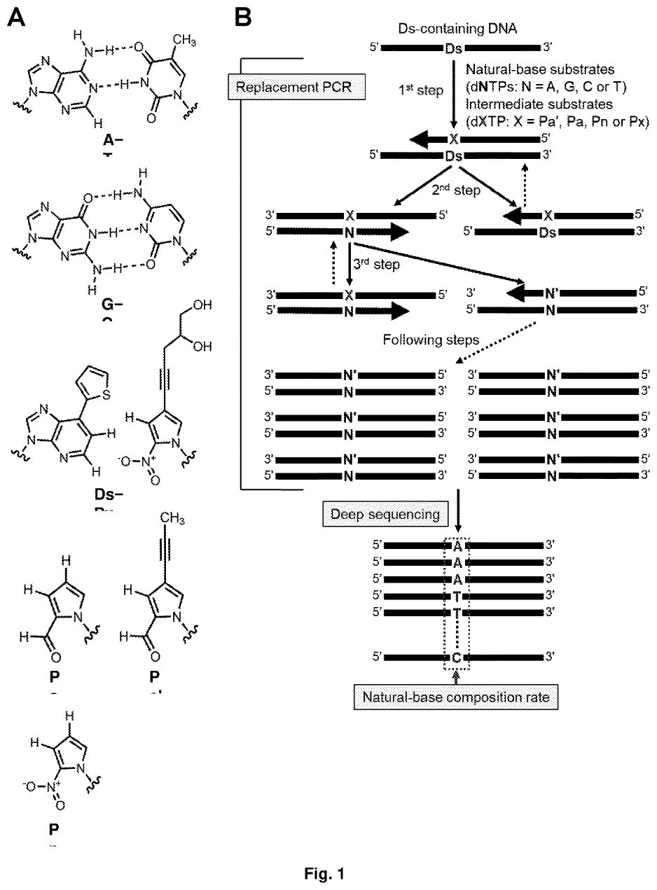

As illustrated in FIG. 1B, the purpose of the replacement replication reaction is to ultimately replace the unnatural base pair with a natural base pair (such that sequencing can be performed on the nucleic acid of interest). Thus, in each one of a replacement replication reaction, the method may comprise the steps of (a) performing a first nucleic acid replication reaction using a first replication substrate containing an intermediate of the unnatural base pair to thereby replace the unnatural base pair with the intermediate of the unnatural base pair; and (b) performing a second nucleic acid replication reaction using a second replication substrate containing natural base pair to thereby replace the intermediate of the unnatural base pair with a natural base pair.

For avoidance of doubt, if two replacement replication reactions are performed, the replacement replication reactions may include the following steps (a) performing a first nucleic acid replication reaction using a first replication substrate containing a first intermediate of the unnatural base pair to thereby replace the unnatural base pair with the first intermediate of the unnatural base pair; (b) performing a second nucleic acid replication reaction using a second replication substrate containing natural base pair to thereby replace the first intermediate of the unnatural base pair with a natural base pair, (c) performing a third nucleic acid replication reaction using a third replication substrate containing a second intermediate of the unnatural base pair to thereby replace the unnatural base pair with the second intermediate of the unnatural base pair; (d) performing a fourth nucleic acid replication reaction using a fourth replication substrate containing natural base pair to thereby replace the second intermediate of the unnatural base pair with a natural base pair. It would be understood that steps (a) to (b) and (c) to (d) are sequential steps. That is, step (a) is to be followed by step (b) and step (c) is to be followed by step (d). However, (a) to (b) and (c) to (d) can be performed separately, concurrently or together. That is, (a) to (b) can be performed at the same time but in a different reaction as (c) to (d).

In some examples, the replacement replication reaction may further comprise replicating or amplification of the nucleic acid from the second nucleic acid replication reaction to thereby have a plurality of nucleic acid with natural base pair resulting from the second nucleic acid replication reaction. This replicating or amplification step is to assist the sequencing of the nucleic acid that has been processed through the replacement PCR.

In some examples, the sequencing may be performed using any high-throughput sequencing methods known in the art. For example, the sequencing may be performed using deep sequencing method or any type of conventional next-generation sequencing to handle enormous amounts of reads without cloning process.

In some examples, the identifying the candidate position of the unnatural base pair may comprise aligning the sequenced nucleic acid and determining a position that contains varying nucleobase. As would be understood by the person skilled in the art, the process of clustering and/or alignment of the sequenced nucleic acids to identify the candidate position of the unnatural base may be performed using a data processing device, such as a data processor.

In some examples, the ratio of conversion of the intermediate to each one of a natural base pair at the candidate position of the unnatural base pair is calculated using the formula:

% rA (at position i)=CR(A,i)=S(A,i)/[S(A,i)+S(G,i)+S(C,i)+S(T,i)]×100

where S(n, i) is the read numbers of sequences which has natural base n at position i.

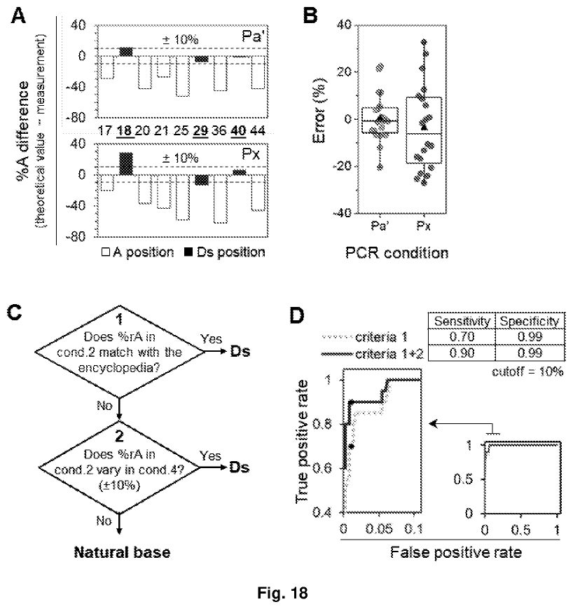

In some examples, a substantial match of the ratio of conversion of the intermediate would result in about 70% or more detection sensitivity, or about 80% or more detection sensitivity, or about 85% or more detection sensitivity, about 90% or more detection sensitivity, or about 91% or more detection sensitivity, or about 92% or more detection sensitivity, or about 93% or more detection sensitivity, or about 94% or more detection sensitivity, or about 95% or more detection sensitivity, or about 96% or more detection sensitivity, or about 97% or more detection sensitivity, or about 98% or more detection sensitivity, or about 99% or more detection sensitivity. In some examples, the substantial match of the ratio of conversion of the intermediate is a value that is not more than (or less than) about 1%, or not more than (or less than) about 2%, or not more than (or less than) about 3%, or not more than (or less than) about 4%, or not more than (or less than) about 5%, or not more than (or less than) about 6%, not more than (or less than) about 7%, or not more than (or less than) about 8%, or not more than (or less than) about 9%, or not more than (or less than) about 10% of the value in the library of the pre-determined conversion/composition rate. In some examples, the substantial match is calculated based on the % rA difference/deviation. In some examples, the % rA difference/deviation may be calculated based on the difference between the value in the library of a pre-determined conversion/comparison rate and the ratio of conversion of the intermediate/actual value from replacement PCR (see for example in FIG. 18A).

In some examples, wherein a substantial match of the ratio of conversion of the intermediate to a value in the library of the pre-determined conversion/composition rate is not achieved, the position of the unnatural base pair may be determined by comparing the ratio of conversion of a first intermediate with the ratio of conversion of a second intermediate. In such examples, an acceptable deviation/difference of the ratio of conversion of a first intermediate from the ratio of conversion of a second intermediate would result in about 90% or more detection sensitivity, or about 91% or more detection sensitivity, or about 92% or more detection sensitivity, or about 93% or more detection sensitivity, or about 94% or more detection sensitivity, or about 95% or more detection sensitivity, or about 96% or more detection sensitivity, or about 97% or more detection sensitivity, or about 98% or more detection sensitivity, or about 99% or more detection sensitivity. In such examples, a varying ratio of conversion of a first intermediate differs from the ratio of conversion of a second intermediate indicates and/or confirms the position of the unnatural base pair. In such example, the varying ratio of conversion of a first intermediate to the ratio of the second intermediate (i.e. % deviation/difference) is a value that is not more than about 10%, or nor more than about 9%, or not more than about 8%, or not more than about 7%, or not more than about 6%, or not more than about 5%, or not more than about 4%, or not more than about 3%, or not more than about 2%, or not more than about 1% of one value to another. In some examples, the varying difference may be calculated using the formula:

VR(i)=|CRp(A,i)−CRq(A,i)|

where CRp(A, i) is the composition rate of a first intermediate to natural base A at position CRq(A, i) is the composition rate of a second intermediate to natural base A at position i, and VR(i) is % deviation/difference at position i.

In another aspect of the present invention, there is provided an apparatus for performing the methods as described herein. For example, the apparatus may include a device for performing the replacement replication reaction (such as a PCR). In some examples, the apparatus may include a device for performing the data clustering, the data point management, and/or data comparison as required in the methods as described herein. In some examples, the apparatus may be an integrated device having all the components required for preforming the methods as described herein.

In some examples, there is provided an apparatus for sequencing a nucleic acid containing an unnatural base pair (UBP), wherein the apparatus comprises a system or device configured to perform one or more replacement replication reaction; a system or device configured to sequence the nucleic acid resulting from the replacement replication reaction; a system or device configured to cluster the sequenced nucleic acid; a system or device configured to identify a candidate position of the unnatural base pair; a system or device configured to determine a ratio of conversion of the intermediate to each one of the natural base pair at the candidate position of the unnatural base pair; a system or device configured to compare the ratio of conversion of the intermediate to a library of pre-determined conversion/composition rate based on the sequences of one or more natural base pair adjacent to the candidate position of the unnatural base pair; and/or a system or device configured to determine the deviation/difference between the ratio of conversion of the intermediate to a value in the library of the pre-determined conversion/composition rate confirms the position of the unnatural base pair, thereby determining the sequence of the nucleic acid containing the unnatural base pair.

It will be appreciated by a person skilled in the art that other variations and/or modifications may be made to the specific embodiments without departing from the scope of the invention as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

### EXPERIMENTAL SECTION

### Materials and Methods

### Reagents and Materials

UB triphosphate substrates (dPxTP (Diol1-dPxTP), dPaTP and dPa′TP) for PCR and dDs-CE-phosphoramidite were chemically synthesized, as described previously (5,8,24,26,27). DNA libraries containing Ds (NDsN2-49 and NDsN3-49, Table 1) were prepared by the conventional phosphoramidite method with an H-8-SE DNA/RNA Synthesizer (K&A Laborgeraete). DNA primers were purchased from Gene Design and Integrated DNA Technologies, or chemically synthesized. DNAs were purified by denaturing gel electrophoresis. Taq DNA polymerase (pol) and AccuPrime Pfx DNA pol were purchased from New England Biolabs and Life Technologies, respectively.

TABLE 1
DNA libraries and PCR primers used in this study.
To analyse the natural-base replacement patterns
at Ds in replacement PCR, the present disclosure
used DNA libraries, NDsN2-49 and NDsN3-49, which
contain randomized regions of the total of four
and six natural bases surrounding one Ds base in
the centre, together with each primer set
(maP25-013/maP25-010 and maP25-011/maP25-10)
for PCR. To validate the developed UB-DNA
sequencing method, the present disclosure used
two enriched DNA libraries in the final round
of ExSELEX: one is for anti-IFNγ UB-DNA aptamer
generation (1) and the other is for anti-vWF
UB-DNA aptamer generation (2). Replacement PCR
was performed by using each enriched DNA library
(N43Ds-P001 mix or N30Ds-S6-006) as the template,
with each primer set (T-27CTT/Rev43.29AA or
mkP25-006/mkP25-009). The initial N43Ds-P001
mix library contained one to three Ds bases
at predetermined positions, which can be
assigned through each natural-base tag
sequence in each sub-library (1).

| | | 5′-Sequence-3′ |
|---|---|---|
| | | (n = A, T ,G, C; |
| | | N = A, T, G, C, Ds; |
| | | XX or XXX: natural- |
| | | base tag sequence in |
| Name | Sample | sub-libraries) |
| NDsN | Library | TAGCGCATAGGTGGGATG |
| 2-49 | | ATGTnnDsnnGTCA |
| | | GATACAATCCTGATCCAT |
| NDsN | Library | ATCCTCACCGATGTACTG |
| 3-49 | | ATGnnnDsnnnTCA |
| | | GATACAATCCTGATCCAT |
| maP2 | Primer | CTATCACTAGCGCATAGGTG |
| 5-013 | (for | GGATG |
| | NDsN2- | |
| | 49) | |
| maP2 | Primer | AGTCTCCATCCTCACCGATG |
| 5-011 | (for | TACTG |
| | NDsN3- | |
| | 49) | |
| maP2 | Primer | CCGTCTCATGGATCAGGAT |
| 5-010 | (common) | TGTATC |
| N43Ds- | Pre- | CTGTCAATCGATCGTATCA |
| P001 | deter- | GTCCACXXnnnnnnnnnnn |
| mix | mined | Nnnnnnnnnnnnnnnnnnn |
| | Ds- | nnnnnnnnnnnnnGCATGA |
| | Library | CTCGAACGGATTAGTGACT |
| | | ACCTGTCAATCGATCGTAT |
| | | CAGTCCACXXXnnnnnnnn |
| | | Nnnnnnnnnnnnnnnnnnn |
| | | nnnnnnnnnnnnnnnnGCA |
| | | TGACTCGAACGGATTAGT |
| | | GACTAC |
| T-27C | Primer | TTCTGTCAATCGATCGTAT |
| TT | | CAGTCCAC |
| Rev43. | Primer | AAGTAGTCACTAATCCGTT |
| 29AA | | CGAGTCATGC |
| N30Ds- | Random | ATCCGCCATACTTACGTTG |
| S6-006 | Ds- | TCCGTGACCNNNNNNNN |
| | Library | NNNNNNNNNNNNNNNN |
| | | NNNNNNGGTCACGTTG |
| | | GAATCTTAAGTGAAGTCG |
| mkP25- | Primer | TATATCCGCCATACTTAC |
| 006 | | GTTGTCC |
| mkP25- | Primer | GCGCGACTTCACTTAAGA |
| 009 | | TTCCAAC |

Replacement PCR for the Conversion from Ds to Natural Bases

To characterize and optimize the replacement PCR, the present disclosure employed two DNA libraries, NDsN2-49 and NDsN3-49, which contain randomized regions with NNDsNN (where N=A, G, C or T) and NNNDsNNN, respectively. For the demonstration using the actual enriched libraries, the present disclosure used the final round of the DNA libraries for anti-IFNγ aptamer generation (N43Ds-P001 mix, Kimoto et al. (24)) and anti-vWF aptamer generation (N30Ds-S6-006, Matsunaga et al. (12)). The Ds bases in each sequence of the DNA libraries were replaced with natural bases through 12 cycles of PCR amplification without dDsTP, which is two-step cycling [94° C. for 15 sec-65° C. for 3 min 30 sec], after 2 min at 94° C. for the initial denaturation step. PCR (100 μl) was performed by using each library (1 pmol) as the template, with 1 μM of each corresponding primer set (Table 1) and each DNA pol at the manufacturer's recommended concentration (AccuPrime Pfx, 0.05 U/μl; Taq, 0.025 U/μl) in the 1× reaction buffer accompanying each DNA pol. In PCR using AccuPrime Pfx DNA pol, 0.1 mM each dNTP and 0.5 mM MgSO₄ were added to the reaction buffer, and the final concentrations of each dNTP and MgSO₄ were 0.4 mM and 1.5 mM, respectively. In PCR using Taq DNA pol, 0.3 mM of each dNTP was used for the reaction. As an intermediate UB substrate, dPa′TP, dPxTP or dPaTP was further added (0.05 mM final concentration). The inventors of the present disclosure examined six different conditions by changing the DNA pols and UB substrates: AccuPrime Pfx DNA pol in the absence of UB substrate (cond. 1), in the presence of dPa′TP (cond. 2), dPaTP (cond. 3) or dPxTP (cond. 4) and Taq DNA pol in the absence of UB substrate (cond. 5) or in the presence of dPa′TP (cond. 6).

### Deep Sequencing

The amplified DNAs obtained by replacement PCR were purified with a QIAquick Gel Extraction Kit (QIAGEN) and sequenced with the IonPGM sequencing system (Life Technologies), according to the manufacturers' instructions. Adapter sequences were ligated to the amplified DNAs using an Ion Plus Fragment Library Kit, and emulsion PCR was performed on a Life Technology OneTouch 2 instrument with the Ion PGM Hi-Q or Hi-Q View OT2 Kit. Enriched template beads were loaded on Ion PGM chips and sequenced with an Ion PGM Hi-Q or Hi-Q View Sequencing Kit. The list of the chips used and the obtained sequencing reads are summarized in Table 2.

TABLE 2
Summary of the sequence reads obtained in this study.

| Replacement PCR | | | Sequencing reads | | | |
|---|---|---|---|---|---|---|
| Template | | Ion PGM ™ | (after | Extracted | Family 1 (Top) | |
| (Library) | UB/Pol | chip | automated QC) | Reads | aptamer clones | |
| NDsN2-49 | —/AccuPrime | 318 ™ v2 | 2,794,237 | 914,954 | | |
| | Pa′/AccuPrime | 318 ™ v2 | 2,412,191 | 761,722 | | |
| | Pa/AccuPrime | 318 ™ v2 | 842,935 | 365,512 | | |
| | Px/AccuPrime | 318 ™ v2 | 912,112 | 424,025 | | |
| | —/Taq | 318 ™ v2 | 1,172,895 | 125,593 | | |
| | Pa′/Taq | 318 ™ v2 | 987,105 | 520,286 | | |
| NDsN3-49 | Pa′/AccuPrime | 318 ™ v2 | 2,591,345 | 720,296 | | |
| | | 318 ™ v2 BC | 3,989,903 | 1,426,530 | | |
| | | 318 ™ v2 BC | 3,442,538 | 1,051,832 | | |
| | Px/AccuPrime | 318 ™ v2 BC | 4,367,087 | 1,710,067 | | |
| | | 318 ™ v2 BC | 4,301,614 | 1,890,147 | | |
| | | 318 ™ v2 BC | 4,033,998 | 1,727,282 | | |
| N43Ds-P001 | Pa′/AccuPrime | 314 ™ v2 | 234,297 | 181,302 | 94,919 | (52%) |
| mix | | 314 ™ v2 | 296,718 | 222,656 | 119,586 | (54%) |
| | | 314 ™ v2 | 242,132 | 176,920 | 93,023 | (53%) |
| | Px/AccuPrime | 314 ™ v2 | 359,165 | 256,097 | 101,093 | (40%) |
| | | 314 ™ v2 | 207,611 | 150,469 | 60,246 | (40%) |
| | | 314 ™ v2 | 287,559 | 195,046 | 74,203 | (38%) |
| N30Ds-S6-006 | Pa′/AccuPrime | 314 ™ v2 | 39,114 | 9,778 | 8.546 | (87%) |
| library #1 | | 314 ™ v2 | 19,663 | 3,190 | 2,721 | (85%) |
| | Px/AccuPrime | 314 ™ v2 | 31,224 | 3,147 | 2.425 | (77%) |
| | | 314 ™ v2 | 60,088 | 3,217 | 2,361 | (73%) |
| N30Ds-S6-006 | Pa′/AccuPrime | 314 ™ v2 | 25,403 | 10,101 | 9,299 | (92%) |
| library #4 | | 314 ™ v2 | 15,124 | 3,247 | 2,978 | (92%) |
| | Px/AccuPrime | 314 ™ v2 | 39,416 | 8,458 | 6,511 | (77%) |
| | | 314 ™ v2 | 17,088 | 1,531 | 1,010 | (66%) |

The Ds bases in each DNA library were replaced with the natural bases under the indicated replacement PCR conditions and analyzed with an IonPGM system using the indicated sequencing chips. Sequencing reads after automated QC and extracted reads after primer sequence trimming (see Materials and Methods) are also indicated. For the N43Ds-P001 mix and N30Ds-S6-006 libraries, the numbers of each target top-ranked aptamer clones (Family 1 sequences, with the percentage against the extracted reads) are indicated in the last column.

### Sequence Data Analysis of NDsN2-49 and NDsN3-49

Sequences were extracted from the deep sequencing data with the following criteria: 5′-(full sequence of the forward primer)-[N bases (N=1-20)]-(complementary sequence of the last six bases of the reverse primer)-3′. The extraction was performed against the complementary sequences as well. The total of both extracted sequences was defined as the “total read counts”. The sequences containing the constant region, 5′-ATGT-(5 bases)-GTCA-3′ for NDsN2-49 and 5′-ATG-(7 bases)-TCA-3′ for NDsN3-49, were retained for further analysis. The composition rates (%) of each natural base converted from Ds (% rN, N=A, T, G, and C) were determined for all of the sequence contexts around Ds (total 4⁴ sequences for NDsN2-49 and 4⁶ for NDsN3-49). For easy comparison across samples, the read count for each sequence context was normalized to reads per million (RPM). For NDsN3-49, replacement PCR reactions with AccuPrime Pfx DNA pol and dPa′TP (cond. Pa′, equal to cond. 2) or dPxTP (cond. Px, equal to cond. 4), as well as the following sequence analyses, were performed in triplicate to calculate the average and variability. The averaged % rN values obtained by this sequencing were employed in the encyclopedia data.

### Sequence Data Analysis Using Enriched Libraries Obtained by ExSELEX

At first, the deep sequencing data were obtained using the N43Ds-P001 mix and N30Ds-S6-006 libraries that were isolated by ExSELEX targeting interferon-γ (IFNγ) and von Willebrand factor A1-domain (vWF), respectively. The sequences were extracted with the following criteria: 5′-(full sequence of the forward primer)-[45 bases (N43Ds-P001 mix) or 42 bases (N30Ds-S6-006)]-(complementary sequence of the last six bases of the reverse primer)-3′. Similarly, the complementary sequences were extracted. To simplify the analysis for the N43Ds-P001 mix libraries, only the aptamer sequences containing the two-base tag (2 bases+43 randomized bases) were extracted. Next, the extracted sequences were clustered into 10-20 families based on the sequence similarities, using in-house Perl scripts (clustered into the same family if the mismatch between the sequence and the top sequence is less than six). Analyses of the N43Ds-P001 libraries were performed in triplicate, and those of the N30Ds-S6-006 libraries were performed twice, to confirm the reproducibility. The obtained % rN values were then compared with the values in the encyclopedia.

### Receiver Operating Characteristic (ROC) Curve Analysis

The sensitivity and selectivity of the sequencing method in the present disclosure were evaluated by a ROC analysis. The use of % rA of the encyclopedia in the anti-IFNγ aptamer selection (criteria 1, see FIG. 18) was validated for a total of 20 Ds bases at predetermined positions in the top ten families of aptamer sequences, by gradually increasing the acceptable range of the deviation between the values in the encyclopedia (reference values) and the selection libraries (actual values). When the deviation is beyond each acceptable value in criteria 1, criteria 2 are also used, where the % rA variation between the data obtained by two replacement PCRs with dPa′TP and dPxTP is more than 10%. The sensitivity (true positive rate) and the specificity (1—false positive rate) were calculated when the acceptable error range for criteria 1 was ±10%.

### Results

### Making an Encyclopedia of Natural-Base Composition Rates by Replacement PCR for all of the Sequence Contexts Around Ds

The composition rates of the natural bases converted from Ds by replacement PCR greatly depend on the natural base sequence contexts around Ds. To simultaneously determine the natural-base composition rates for all of the sequence contexts, the present study used DNA libraries containing natural-base randomized sequences and Ds (FIG. 2). The inventors of the present disclosure chemically synthesized two DNA libraries, NDsN2-49 and NDsN3-49, containing the random regions, NNDsNN (4⁴=256 combinations, N=A, G, C or T) and NNNDsNNN (4⁶=4,096 combinations), respectively (Table 1). First, NDsN2-49 was used to optimize the replacement PCR conditions, in the absence or presence of intermediate UB substrates, such as dPa′TP, dPaTP, and dPxTP, using AccuPrime Pfx or Taq DNA pol. Next, the data was obtained to make an encyclopedia of the natural base replacement (ENBRE), using NDsN3-49.

The amplified double-stranded DNAs after 12 cycles of replacement PCR were subjected to deep sequencing with the IonPGM system. All of the extracted sequences with the correct length were classified into each sequence context around Ds, and the natural-base composition rates at the initial Ds position were determined in each sequence context. The data were then compiled as the encyclopedia, ENBRE (FIG. 2). To evaluate the accuracy of this sequencing method, ENBRE was compared with the actual sequencing data obtained from replacement PCR, using the enriched libraries after the ExSELEX procedures.

### Intermediate UB Substrates for Replacement PCR

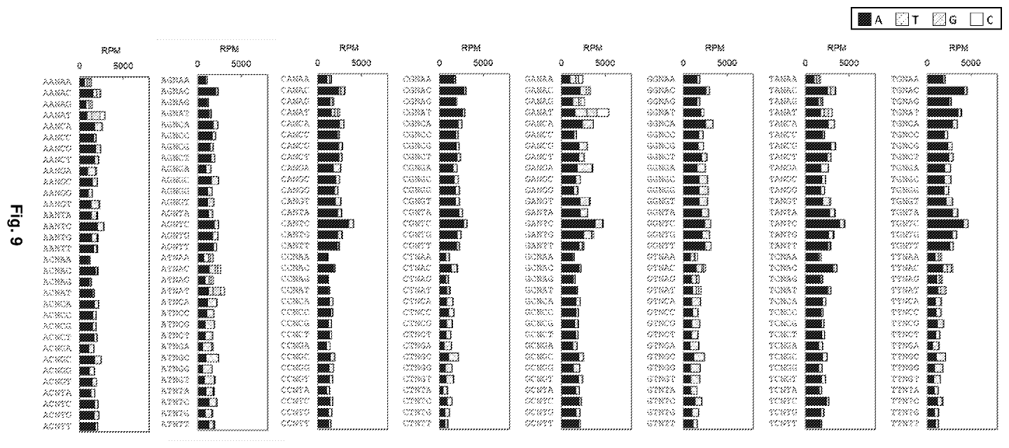

First, the replacement PCR of the NNDsNN library was examined using AccuPrime Pfx DNA pol without any intermediate UB substrates (FIG. 3A, the left flow) and collected the read counts and the natural-base composition rates at the original Ds position in each sequence context (FIG. 3B and FIG. 8). Due to the high fidelity of the Ds-Px pair in PCR, most of the sequence contexts were difficult to amplify without dDsTP and dPxTP, resulting in low read counts. Interestingly, the NYDsTN (Y=C or T) contexts yielded high read counts, indicating that the Ds bases in NYDsTN were easily mutated to natural bases, mainly to A. In contrast, the natural-base conversions from the Ds bases in NRDsRN (R=A or G) were very hard. These results provided a new perception about the replication of the Ds-Px pair. In PCR involving the Ds-Px pair, the amplification efficiencies of the NRDsRN contexts are lower than those of the NYDsYN contexts. However, the current results indicated a lower risk of the mutation from Ds to natural bases in the NRDsRN contexts than in the NYDsTN contexts during PCR. Thus, DNAs containing the inefficient NRDsRN sequences can be sufficiently amplified by increasing the PCR cycles in the presence of dDsTP and dPxTP, while retaining the low Ds-mutation rates. Indeed, the fidelities of all of sequence contexts were very high (>99.9%/doubling) in PCR using Deep Vent DNA pol (exo+).

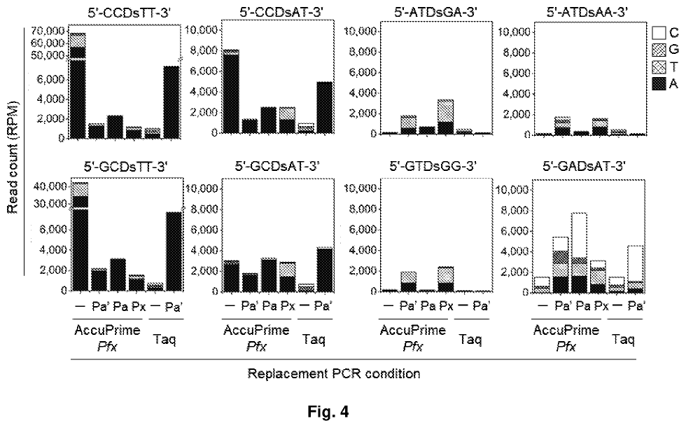

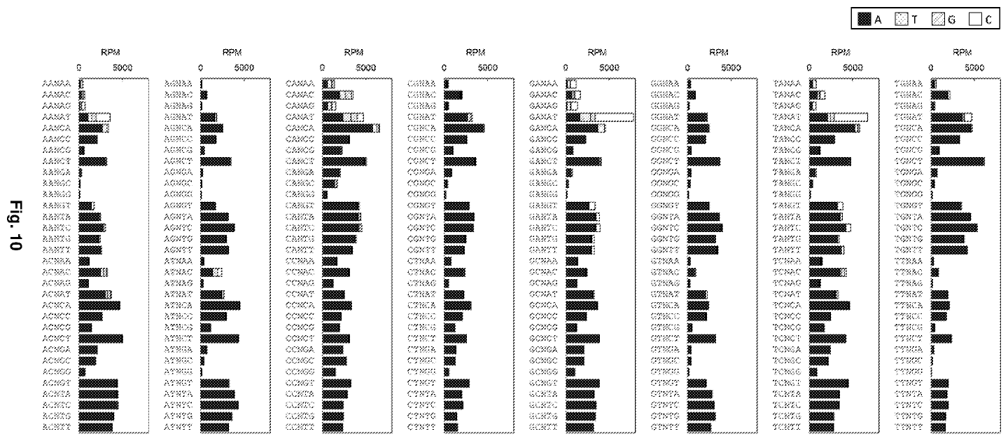

Next, dPa′TP was added as an intermediate substrate for replacement PCR using AccuPrime Pfx DNA pol (FIG. 3A, the right flow). The addition of dPa′TP greatly accelerated the conversion from Ds to natural bases in all of the sequence contexts (FIG. 3C and FIG. 9). The natural-base compositions converted from Ds significantly varied depending on the sequence contexts (FIG. 4). For example, the Ds bases in NCDsTN, NCDsAN, and NGDsAN converted to A>>T>>C≈G. In contrast, the Ds bases in NTDsGN converted to T≥A>>G≈C. The Ds→J conversion might occur through the misincorporation of dTTP opposite Pa′, after the dPa′TP incorporation opposite Ds. Interestingly, the Ds bases in some of the NTDsAN and NADsAN contexts converted to the four natural bases at a nearly equal ratio.

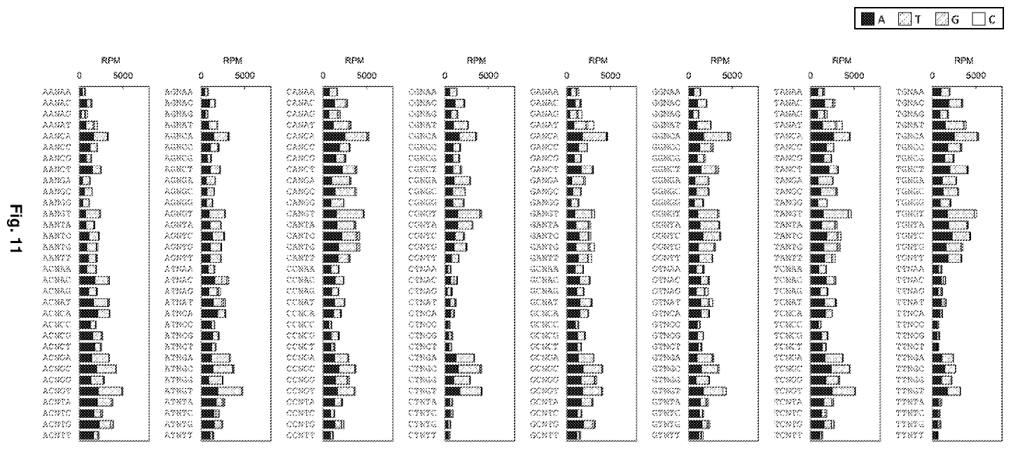

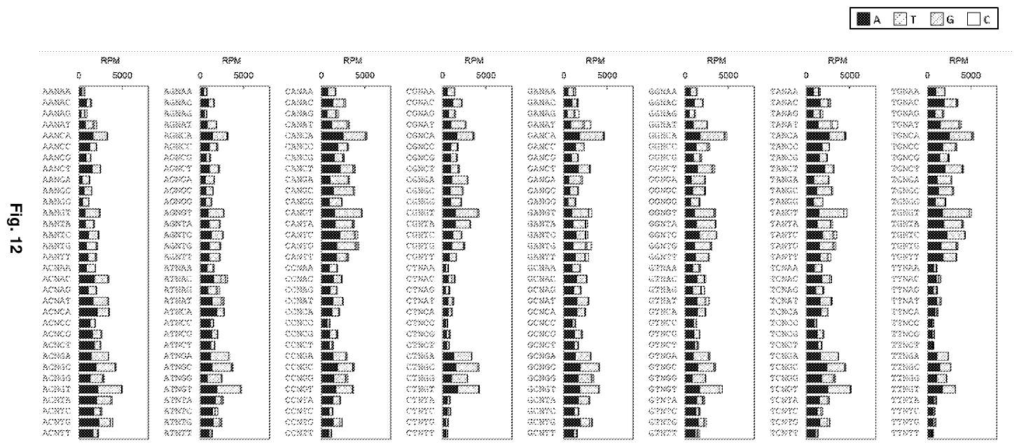

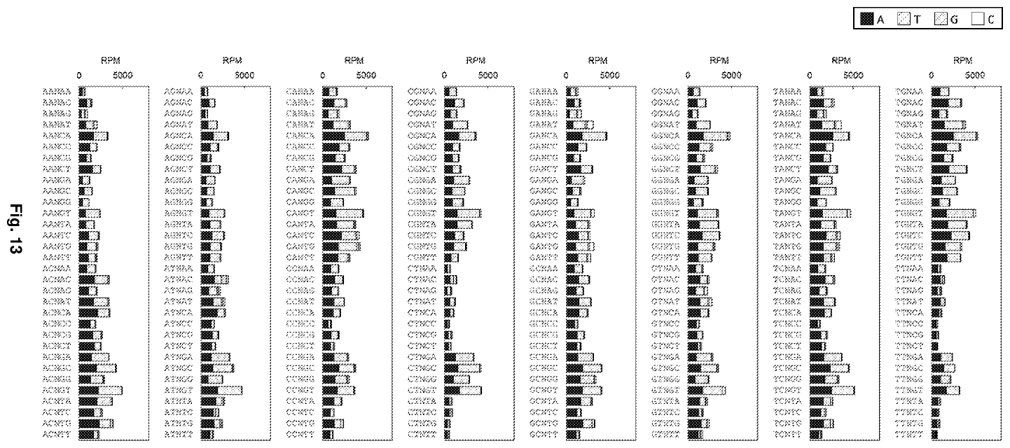

dPaTP (Pa: pyrrole-2-carbaldehyde) and dPxTP were also examined as other UB intermediate substrates for replacement PCR with AccuPrime Pfx DNA pol (FIG. 4, FIG. 10 and FIG. 11). When using dPaTP, the Ds→A conversion became predominant in most of the sequence contexts, except for XADsAT (X=A, G or T) (FIG. 10). This might occur because the efficiency of the Pa incorporation is lower than that of Pa′ in replication, reducing the misincorporation of dTTP opposite Pa in templates more than the dATP misincorporation opposite Pa. In contrast, the dPxTP addition as the intermediate substrate increased the Ds→T conversion, which was as high as the Ds→A conversion (FIG. 11). The oxygen in the nitro group of Px efficiently reduces the Px misincorporation opposite A, as compared to Pa′, due to the electrostatic repulsion between the oxygen of Px and the N1 of A. Thus, instead of the A misincorporation, the T misincorporation opposite Px relatively increased and the composition of the natural bases after replacement PCR with dPxTP changed to A≈T>>C≈G.

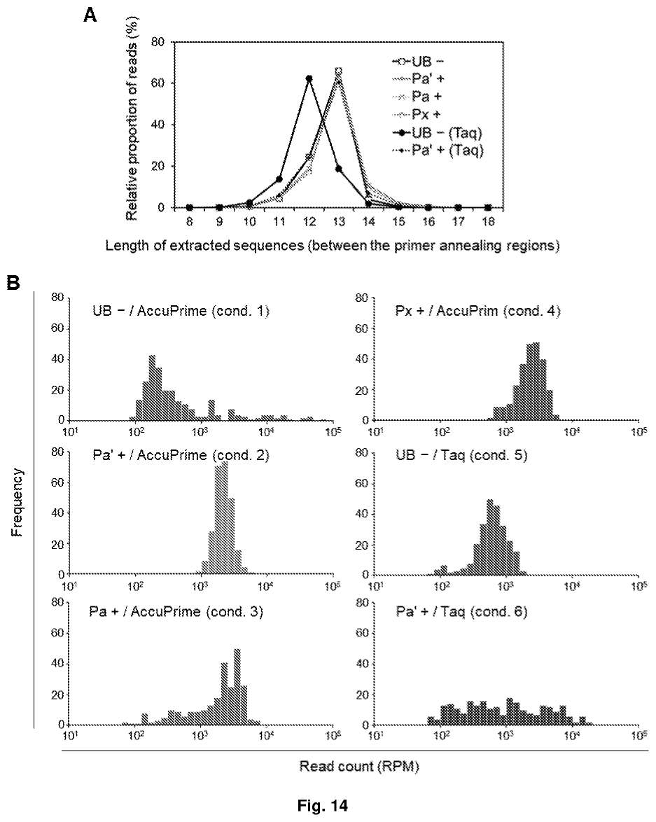

Besides AccuPrime Pfx DNA pol, Taq DNA pol was tested for replacement PCR in the presence and absence of dPa′TP (FIG. 12 and FIG. 13). In previous studies, it was revealed that the fidelity of the Ds-Px pair in replication using Taq DNA pol is much lower than that using AccuPrime Pfx DNA pol, and the Ds-Px pair is easily mutated to natural base pairs by Taq DNA pol in PCR. As expected, the replacement PCR using Taq DNA pol in the absence of any intermediate UB substrates proceeded with most of the sequence contexts (except for NNDsGG) and Ds converted to any natural bases. However, Taq DNA pol was found to produce a one base deletion with high frequency (62%) during replacement PCR (FIG. 14A). In the presence of dPa′TP, Taq DNA pol promoted the Ds→A conversion but increased the bias of the conversion efficiency depending on the sequence contexts (FIG. 13 and FIG. 14B).

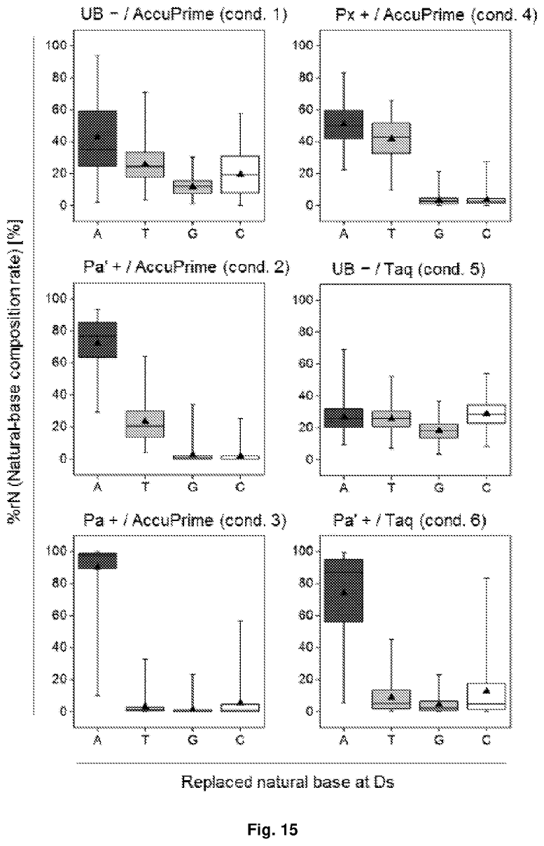

Overall, replacement PCR in the presence of dPa′TP using AccuPrime Pfx DNA pol was the best combination for all of the sequence contexts, and the replacement PCR in the presence of dPxTP was the second best (FIG. 14). After the replacement PCR in each condition, the natural-base compositions rate (% of each natural base) at the Ds position varied depending on the sequence contexts (FIG. 4). In addition, replacement PCR using dPxTP generally increased the Ds→T conversion, as compared to that using dPa′TP (FIG. 15).

### Preparation of Two Sets of Encyclopedias of Replacement PCR for Each Sequence Context (ENBRE)

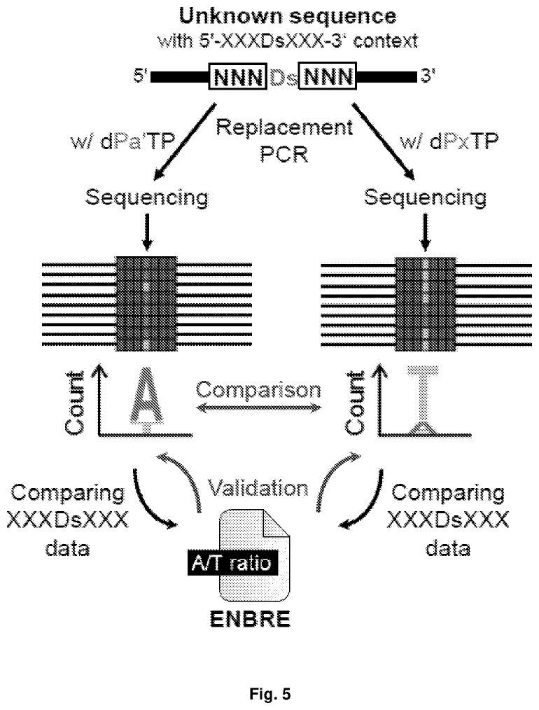

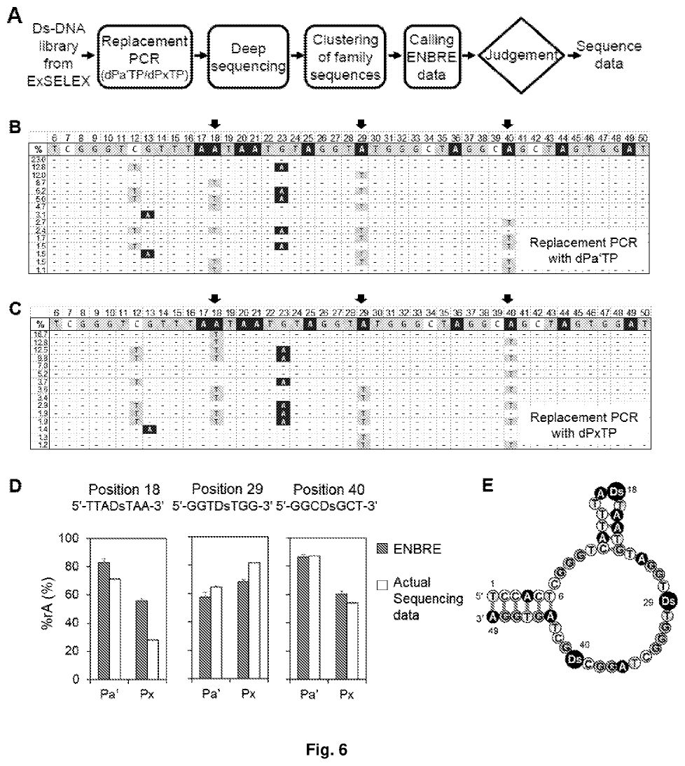

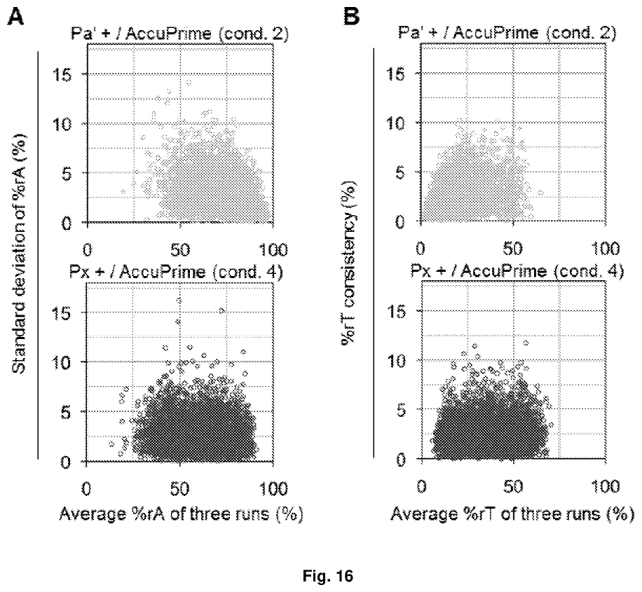

Based on the above results using the NNDsNN library, two sets of the encyclopedias of the natural-base composition rates was prepared for each sequence context in replacement PCR in the presence of either dPa′TP or dPxTP, using NNNDsNNN (4⁶=4,096 combinations) and AccuPrime Pfx DNA pol, to increase the accuracy of ENBRE (FIG. 5). The replacement PCR and sequencing analysis were performed three times independently in each replacement PCR method and confirmed the high reproducibility (approx. <10% S.D.) of the natural base composition rates for each sequence context (FIG. 16). To simplify the searching method using ENBRE, the present study focused on the Ds→A conversion rates (% rA), because the % rA values greatly varied in the range of 19.2-97.5% (in dPa′TP-replacement PCR) (Table 3) depending on the sequence context. In addition, the intermediate substrates, either dPa′TP or dPxTP, also greatly changed the conversion rates in the same sequence contexts. Using the encyclopedia, the Ds positions in each aptamer candidate family can be identified by comparing the % rA values between ENBRE and the actual data obtained by replacement PCR of enriched libraries by each ExSELEX procedure (FIG. 5).

Furthermore, from the difference in the % rA values between the two replacement PCRs with dPa′TP and dPxTP, the present study could confirm the existence of Ds in each aptamer candidate obtained from the final round of ExSELEX. If the mutation from Ds to natural bases occurred during the ExSELEX procedures, then the differences in the % rA values obtained by the two replacement PCRs would not be observed.

Evaluation of the Sequencing Method Using UB-DNA Aptamer Sequences from Enriched Libraries Obtained by ExSELEX

To verify the accuracy of ENBRE, the sequencing method was tested by using two actual enriched libraries, which were obtained by ExSELEX procedures targeting interferon-γ (IFNγ) and von Willebrand factor A1-domain (vWF). From the libraries, high-affinity Ds-containing DNA aptamers were obtained for both targets. The anti-IFNγ aptamer (K_{D}=38 pM) was obtained as one of the first Ds-containing aptamers, using a predetermined library comprised of ˜20 sub-libraries. The aptamer contained three Ds bases, and two Ds bases were essential for the tight binding to IFNγ. Previously, the Ds positions in the aptamer sequence were determined using the specific barcode that was embedded into each sub-library. The anti-vWF aptamer (K_{D}=75 pM) was obtained by ExSELEX using six different batches (#1-#6) of the chemically synthesized DNA library with randomized sequences including Ds bases. The inventors of the present disclosure previously obtained two aptamer families from libraries #1 and #4 and determined the Ds positions in each aptamer family by modified Sanger sequencing using each aptamer candidate, which was isolated by hybridization with a specific probe from the enriched library.

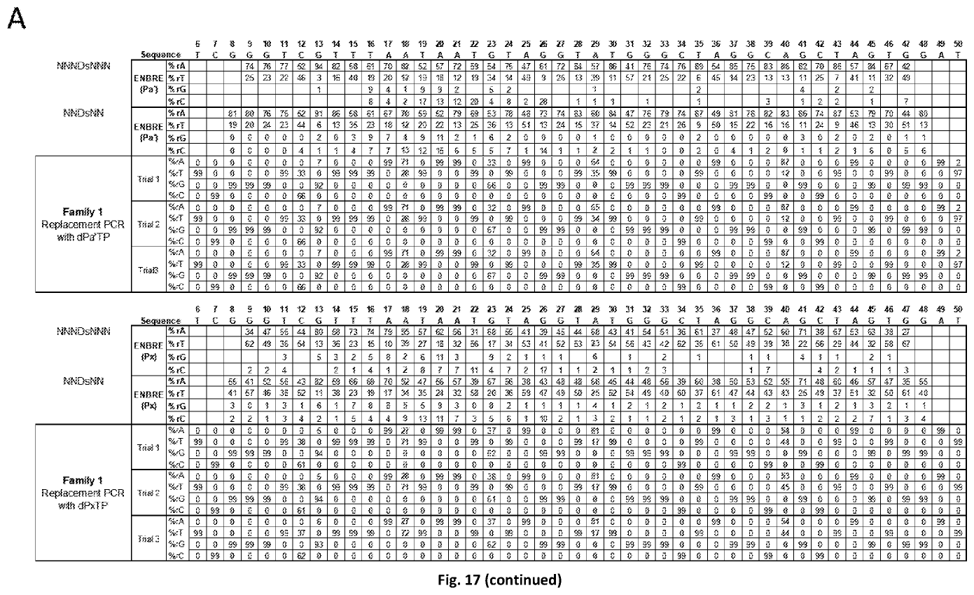

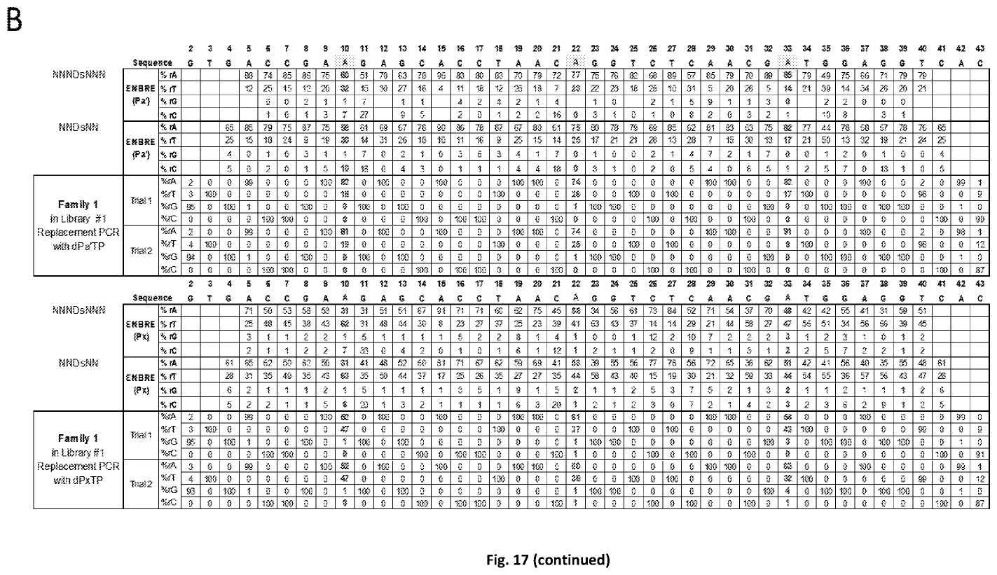

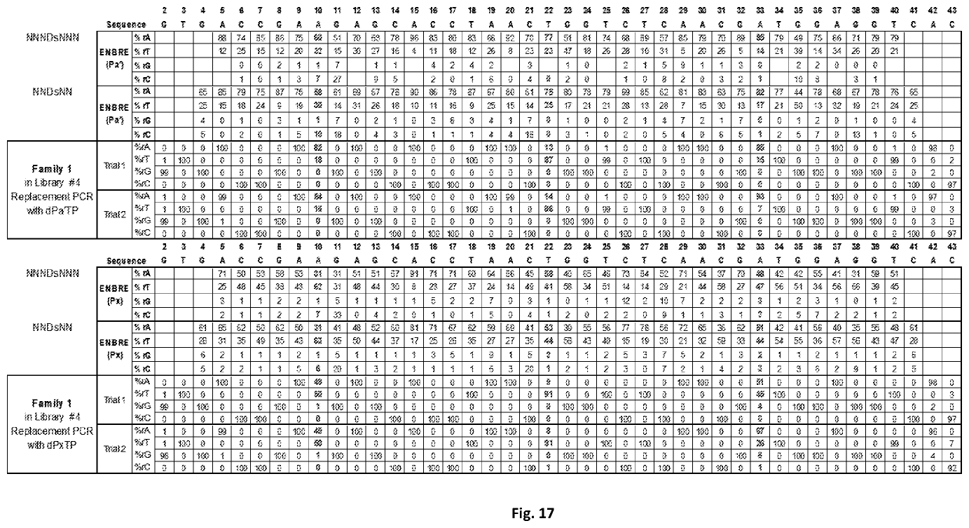

FIG. 6A shows the sequencing procedure. First, two replacement PCR methods was performed in the presence of either dPa′TP or dPxTP (Step a). Second, natural-base sequence data was obtained by deep sequencing, using the Ion PGM system (Step b, Table 2). Third, both of the sequence data sets obtained using dPa′TP and dPxTP were aligned and clustered to find each family of clones (Step c). Fourth, the % rA values (or the natural-base composition rates) of each position in the family sequence were compared with the ENBRE data (Step d, FIG. 17). If the % rA values of each position were similar to those in ENBRE, then these positions were concluded to be corresponded to the Ds positions in the original candidate sequence (Step e).

First, to analyze the sequences of the anti-IFNγ aptamer, replacement PCR was performed in the presence of dPa′TP or dPxTP using the enriched library (N43Ds-P001 mix in Table 1) that was previously obtained after seven rounds of ExSELEX (11) (FIG. 6). Among the total sequences, approximately 50% of the sequences (family 1) were enriched to the anti-IFNγ aptamer sequence (FIG. 6E and Table 2). The % rA values at each position in the total sequences of family 1 were scanned by comparison with the rates of the ENBRE data (FIG. 17A), and it was found that the rates at three positions, 18, 29, and 40, were close (<10% deviation in the Ds→A conversion rates) to those of the ENBRE data (FIGS. 6B, 6C, and 6D). One exception was the value at position 18 obtained by replacement PCR with dPxTP, which showed approx. 30% deviation and the % rA of the experimental data was much lower than that in ENBRE (FIG. 18A). This difference might indicate that position 18 in the enriched library would be a mixture of the Ds and natural T bases. Since the Ds base at position 18 is not essential for the binding to IFNγ, the Ds base might be mutated to natural bases during the ExSELEX procedure.

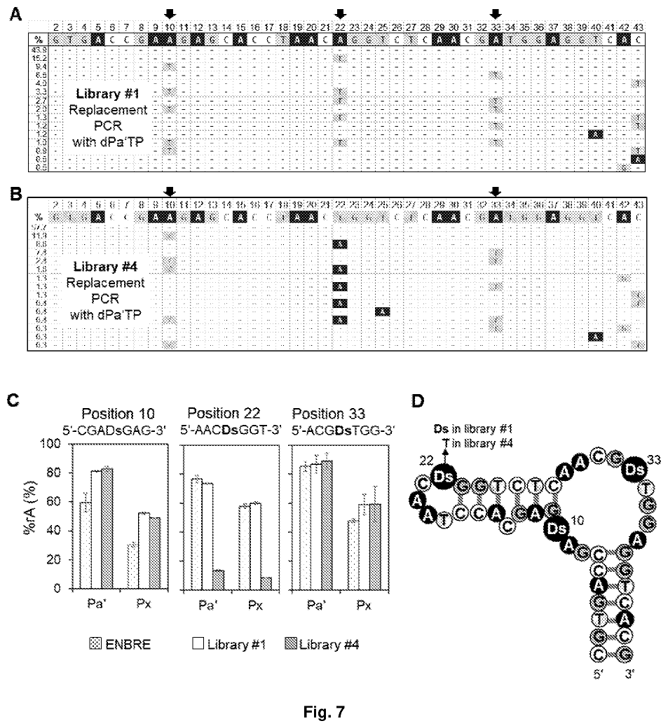

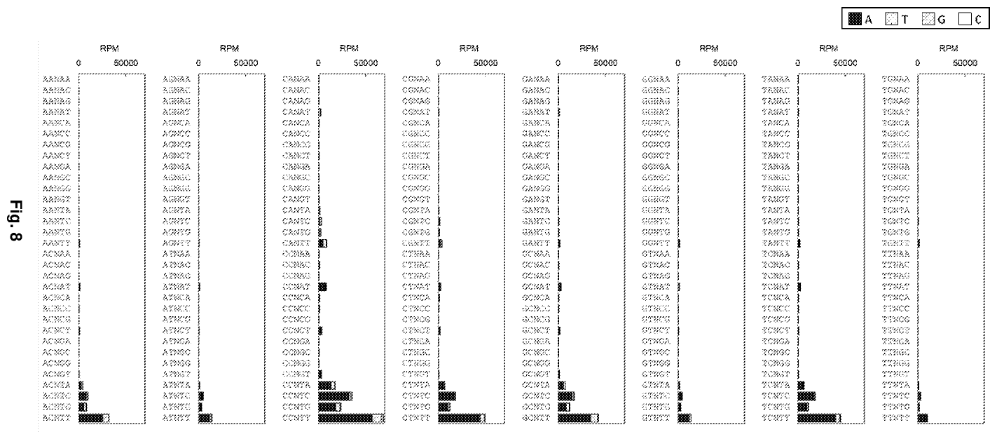

Next, two enriched libraries #1 and #4 obtained by ExSELEX targeting vWF was analyzed using the Ds-randomized library (12) (FIG. 7). The main family sequences from #1 and #4 were mostly identical, except for one Ds position (position 22): the one obtained from #1 contained three Ds bases at positions 10, 22, and 33 and the other from #4 contained two Ds bases at positions 10 and 33 (FIG. 7D). The Ds base at position 22 in the aptamers was not essential for the tight binding to vWF (12). Here, replacement PCR were performed using libraries #1 and #4 and aligned the top clustered sequences (FIGS. 7A and 7B, FIG. 17B). The % rA value at position 22 from #4 was significantly different (>50% deviation) between the actual and ENBRE data (FIG. 7C, FIG. 17B). In addition, the natural-base composition rates at position 22 from #4 were identical between those obtained by the two replacement PCR methods with either dPa′TP or dPxTP (FIG. 17B). Thus, the base at position 22 from #4 was identified as the natural bases (mostly T), rather than Ds. Besides position 22, the % rA values at position 10 from #1 and #4 were deviated from those in the ENBRE data (>20% deviation). This might be because the Ds bases at position 10 in the families were partially mutated to A during the PCR amplification in the seven rounds of selection (157 PCR cycles in total) or because the isolated libraries after the first round already contained the natural base species, instead of Ds. This possibility was supported by the gel-shift assay of the vWF-aptamer complex, where the vWF-binding efficiencies using the enriched libraries were very low as compared to those using the chemically synthesized Ds-containing aptamers corresponding to families #1 and #4 (12). However, the % rA values at position 10 were quite different between the two replacement PCR methods with either dPa′TP or dPxTP, and thus the present disclosure concluded that the Ds base still existed at position 10 in most of the DNAs.

To assess the accuracy of the ENBRE data for DNA sequencing involving Ds bases, the present study broadly explored the % rA values of the sequencing data for the anti-IFNγ aptamer generation, in which the library containing Ds bases was used at defined positions. The differences of the % rA values between the actual data of the enriched library and the ENBRE data was analysed using 20 Ds positions in the top ten families of the anti-IFNγ aptamer sequences (FIG. 18). For both of the replacement PCR methods using dPa′TP or dPxTP, the means of the deviations of the % rA values were close to 0. However, some outliers appeared with relatively higher errors (especially in the replacement PCR using dPxTP). Thus, when using <10% deviation of the % rA values obtained by replacement PCR using dPa′TP as the initial criterion for the detection of the Ds positions, the sensitivity is 0.70 (FIGS. 18C and D). To increase the sensitivity, the additional criterion using the two replacement PCR methods with either dPa′TP or dPxTP was employed. If the deviation is larger than 10% in the first step, then the use of the second criterion, which is >±10% fluctuation between two replacement PCR methods, could improve the sensitivity by 0.90 without any loss of specificity (FIG. 18).

To develop a sequencing method for Ds-DNA aptamer generation, the replacement PCR method was optimised, and it was found that the two replacement PCR methods using AccuPrime Pfx DNA pol and either dPa′TP or dPxTP as an intermediate substrate efficiently convert Ds to natural bases in the amplified DNAs. The natural-base composition rates converted from Ds significantly varied, depending on the use of the intermediate substrates and the sequence contexts around Ds. Thus, two ENBRE databases were made corresponding to all of the sequence contexts for both dPa′TP- and dPxTP-replacement PCRs. In general, replacement PCR with dPa′TP converts Ds to A>>T>>C≈G in most of the sequence contexts. In contrast, replacement PCR with dPxTP increased the conversion rates from Ds to T, as compared with that with dPa′TP. These differences in the conversion tendencies between the two intermediate substrates increased the accuracy for the determination of the Ds positions in the Ds-DNA aptamer candidate sequences.

This approach facilitates the deep sequencing method to identify a single clone containing Ds bases from enriched libraries containing different sequences obtained by ExSELEX. The present disclosure has demonstrated the DNA sequencing of Ds-DNA aptamer candidates in the enriched libraries obtained by ExSELEX targeting IFNγ and vWF. This sequencing method could simplify the process and thus shorten the time required for Ds-DNA aptamer generation using libraries with randomized sequences containing Ds. In addition, besides the Ds-Px pair, this method could be applied to other unnatural base pair systems.

This study also provides valuable information about replication fidelity involving UBPs. The replacement PCR in the absence of intermediate UB-substrates greatly reduced the conversion efficiency from Ds to natural bases. This fact confirmed the high fidelity of the Ds-Px pair in replication. In addition, these data are useful to design an efficient Ds-containing sequence context for replication. For example, the replacement PCR in the absence of intermediate UB-substrates predominantly replaced Ds in the NYDsTN sequence contexts with natural bases, but was not efficient for Ds in the NYDsCN sequence contexts. Since both of the NYDsTN and NYDsCN sequence contexts exhibited high efficiency in PCR amplification, the NYDsCN sequence contexts among them might exhibit the highest efficiency and fidelity in PCR. Furthermore, the present disclosure found that each sequence context yielded varied natural-base composition rates by replacement PCR with dPa′TP. In particular, the NADsAN or NTDsAN sequence contexts tended to increase the misincorporation of dGTP and dCTP opposite Ds. This indicated that the Ds conformation in such sequences might be different from those in other sequences within the polymerase active site. Furthermore, the present disclosure found that Taq DNA pol (family A pol) caused the deletion mutation during replacement PCR, although AccuPrime Pfx and Deep Vent DNA pols (family B pol) rarely observed such a mutation during PCR in the presence of dDsTP and dPxTP. Since the Ds-Px pair functions in PCR using family B pol, the results using family A pol could provide an insight for UBP replication together with the information of structural data of the ternary complex of KlenTaq DNA poly (family A pol) with a Ds-template/primer duplex bound to dPxTP. These data will be useful for further studies to create improved UBPs with higher fidelity and efficiency.

### REFERENCES

1. Hamashima, K., Kimoto, M. and Hirao, I. (2018) Creation of unnatural base pairs for genetic alphabet expansion toward synthetic xenobiology. Curr. Opin. Chem. Biol., 46, 108-114.
2. Lee, K. H., Hamashima, K., Kimoto, M. and Hirao, I. (2018) Genetic alphabet expansion biotechnology by creating unnatural base pairs. Curr. Opin. Biotechnol., 51, 8-15.
3. Dien, V. T., Morris, S. E., Karadeema, R. J. and Romesberg, F. E. (2018) Expansion of the genetic code via expansion of the genetic alphabet. Curr. Opin. Chem. Biol., 46, 196-202.
4. Karalkar, N. B. and Benner, S. A. (2018) The challenge of synthetic biology. Synthetic Darwinism and the aperiodic crystal structure. Curr. Opin. Chem. Biol., 46, 188-195.
5. Kimoto, M., Kawai, R., Mitsui, T., Yokoyama, S. and Hirao, I. (2009) An unnatural base pair system for efficient PCR amplification and functionalization of DNA molecules. Nucleic Acids Res., 37, e14.
6. Yamashige, R., Kimoto, M., Mitsui, T., Yokoyama, S. and Hirao, I. (2011) Monitoring the site-specific incorporation of dual fluorophore-quencher base analogues for target DNA detection by an unnatural base pair system. Org. Biomol. Chem., 9, 7504-7509.
7. Okamoto, I., Miyatake, Y., Kimoto, M. and Hirao, I. (2016) High fidelity, efficiency and functionalization of Ds-Px unnatural base pairs in PCR amplification for a genetic alphabet expansion system. ACS Synth. Biol., 5, 1220-1230.
8. Yamashige, R., Kimoto, M., Takezawa, Y., Sato, A., Mitsui, T., Yokoyama, S. and Hirao, I. (2012) Highly specific unnatural base pair systems as a third base pair for PCR amplification. Nucleic Acids Res., 40, 2793-2806.
9. Yang, Z., Sismour, A. M., Sheng, P., Puskar, N. L. and Benner, S. A. (2007) Enzymatic incorporation of a third nucleobase pair. Nucleic Acids Res., 35, 4238-4249.
10. Yang, Z., Chen, F., Alvarado, J. B. and Benner, S. A. (2011) Amplification, mutation, and sequencing of a six-letter synthetic genetic system. J. Am. Chem. Soc., 133, 15105-15112.
11. Kimoto, M., Yamashige, R., Matsunaga, K., Yokoyama, S. and Hirao, I. (2013) Generation of high-affinity DNA aptamers using an expanded genetic alphabet. Nat. Biotechnol., 31, 453-457.
12. Matsunaga, K., Kimoto, M. and Hirao, I. (2017) High-affinity DNA aptamer generation targeting von Willebrand factor A1-domain by genetic alphabet expansion for systematic evolution of ligands by exponential enrichment using two types of libraries composed of five different bases. J. Am. Chem. Soc., 139, 324-334.
13. Sefah, K., Yang, Z., Bradley, K. M., Hoshika, S., Jimenez, E., Zhang, L., Zhu, G., Shanker, S., Yu, F., Turek, D. et al. (2014) In vitro selection with artificial expanded genetic information systems. Proc. Natl. Acad. Sci. USA, 111, 1449-1454.
14. Zhang, L., Yang, Z., Sefah, K., Bradley, K. M., Hoshika, S., Kim, M. J., Kim, H. J., Zhu, G., Jimenez, E., Cansiz, S. et al. (2015) Evolution of functional six-nucleotide DNA. J. Am. Chem. Soc., 137, 6734-6737.
15. Zhang, L., Yang, Z., Le Trinh, T., Teng, I. T., Wang, S., Bradley, K. M., Hoshika, S., Wu, Q., Cansiz, S., Rowold, D. J. et al. (2016) Aptamers against cells overexpressing glypican 3 from expanded genetic systems combined with cell engineering and laboratory evolution. Angew. Chem. Int. Ed. Engl., 55, 12372-12375.
16. Biondi, E., Lane, J. D., Das, D., Dasgupta, S., Piccirilli, J. A., Hoshika, S., Bradley, K. M., Krantz, B. A. and Benner, S. A. (2016) Laboratory evolution of artificially expanded DNA gives redesignable aptamers that target the toxic form of anthrax protective antigen. Nucleic Acids Res., 44, 9565-9577.
17. Malyshev, D. A., Seo, Y. J., Ordoukhanian, P. and Romesberg, F. E. (2009) PCR with an expanded genetic alphabet. J. Am. Chem. Soc., 131, 14620-14621.
18. Malyshev, D. A., Dhami, K., Quach, H. T., Lavergne, T., Ordoukhanian, P., Torkamani, A. and Romesberg, F. E. (2012) Efficient and sequence-independent replication of DNA containing a third base pair establishes a functional six-letter genetic alphabet. Proc. Nat. Acad. Sci. USA, 109, 12005-12010.
19. Li, L., Degardin, M., Lavergne, T., Malyshev, D. A., Dhami, K., Ordoukhanian, P. and Romesberg, F. E. (2014) Natural-like replication of an unnatural base pair for the expansion of the genetic alphabet and biotechnology applications. J. Am. Chem. Soc., 136, 826-829.
20. Malyshev, D. A., Dhami, K., Lavergne, T., Chen, T., Dai, N., Foster, J. M., Correa, I. R., Jr. and Romesberg, F. E. (2014) A semi-synthetic organism with an expanded genetic alphabet. Nature, 509, 385-388.
21. Zhang, Y., Ptacin, J. L., Fischer, E. C., Aerni, H. R., Caffaro, C. E., San Jose, K., Feldman, A. W., Turner, C. R. and Romesberg, F. E. (2017) A semi-synthetic organism that stores and retrieves increased genetic information. Nature, 551, 644-647.
22. Dien, V. T., Holcomb, M., Feldman, A. W., Fischer, E. C., Dwyer, T. J. and Romesberg, F. E. (2018) Progress Toward a Semi-Synthetic Organism with an Unrestricted Expanded Genetic Alphabet. J. Am. Chem. Soc., 140, 16115-16123.
23. Ohtsuki, T., Kimoto, M., Ishikawa, M., Mitsui, T., Hirao, I. and Yokoyama, S. (2001) Unnatural base pairs for specific transcription. Proc. Natl. Acad. Sci. USA, 98, 4922-4925.
24. Hirao, I., Kimoto, M., Mitsui, T., Fujiwara, T., Kawai, R., Sato, A., Harada, Y. and Yokoyama, S. (2006) An unnatural hydrophobic base pair system: site-specific incorporation of nucleotide analogs into DNA and RNA. Nat. Methods, 3, 729-735.
25. Hirao, I., Mitsui, T., Kimoto, M. and Yokoyama, S. (2007) An efficient unnatural base pair for PCR amplification. J. Am. Chem. Soc., 129, 15549-15555.
26. Mitsui, T., Kitamura, A., Kimoto, M., To, T., Sato, A., Hirao, I. and Yokoyama, S. (2003) An unnatural hydrophobic base pair with shape complementarity between pyrrole-2-carbaldehyde and 9-methylimidazo[(4,5)-b]pyridine. J. Am. Chem. Soc., 125, 5298-5307.
27. Mitsui, T., Kimoto, M., Sato, A., Yokoyama, S. and Hirao, I. (2003) An unnatural hydrophobic base, 4-propynylpyrrole-2-carbaldehyde, as an efficient pairing partner of 9-methylimidazo[(4,5)-b]pyridine. Bioorg. Med. Chem. Lett., 13, 4515-4518.
28. Betz, K., Kimoto, M., Diederichs, K., Hirao, I. and Marx, A. (2017) Structural basis for expansion of the genetic alphabet with an artificial nucleobase pair. Angew. Chem. Int. Ed. Engl.

1. A method of sequencing a nucleic acid containing an unnatural base pair (UBP), comprising:
performing two or more replacement replication reactions wherein the nucleic acid is replicated using two or more intermediate of the unnatural base pair;
sequencing the nucleic acid resulting from the replacement replication reactions;
clustering the sequenced nucleic acid and identifying a candidate position of the unnatural base pair;
determining a ratio of conversion of the intermediate to each one of a natural base pair at the candidate position of the unnatural base pair; and
comparing the ratio of conversion of the intermediate to a library of pre-determined conversion rate based on the sequences of one or more natural base pair adjacent to the candidate position of the unnatural base pair;
wherein a substantial match of the ratio of conversion of the intermediate to a value in the library of the pre-determined conversion rate confirms the position of the unnatural base pair, thereby determining the sequence of the nucleic acid containing the unnatural base pair.

2. The method of claim 1, wherein the method comprises two replacement replication reactions, optionally the two replacement replication reactions comprise:
performing a first replacement replication reaction wherein the nucleic acid is replicated using a first intermediate of the unnatural base pair; and
performing a second replacement replication reaction wherein the nucleic acid is replicated using a second intermediate of the unnatural base pair, optionally the two replacement reactions are performed concurrently, sequentially, and/or separately, optionally the first intermediate and the second intermediate are different intermediate of an unnatural base pair.

3.-5. (canceled)

6. The method of claim 1, wherein the intermediate of the unnatural base pair is selected from the group consisting of Pa′, Pa, Pn, and Px.

7. The method of claim 1, wherein the unnatural base pair is composed of a nucleobase selected from the group consisting of:
a 7-(2-thienyl)imidazo[4,5-b]pyridin-3-yl group (Ds);
a 7-(2,2′-bithien-5-yl)imidazo[4,5-b]pyridin-3-yl group (Dss);
a 7-(2,2′,5′,2″-terthien-5-yl)imidazo[4,5-b]pyridin-3-yl group (Dsss);
a 2-amino-6-(2-thienyl)purin-9-yl group (s);
a 2-amino-6-(2,2′-bithien-5-yl)purin-9-yl group (ss);
a 2-amino-6-(2,2′,5′,2″-terthien-5-yl)purin-9-yl group (sss);
a 4-(2-thienyl)-pyrrolo[2,3-b]pyridin-1-yl group (dDsa);
a 4-(2,2′-bithien-5-yl)-pyrrolo[2,3-b]pyridin-1-yl group (Dsas);
a 4-[2-(2-thiazolyl)thien-5-yl]pyrrolo[2,3-b]pyridin-1-yl group (Dsav);
a 4-(2-thiazolyl)-pyrrolo[2,3-b]pyridin-1-yl group (dDva);
a 4-[5-(2-thienyl)thiazol-2-yl]pyrrolo[2,3-b]pyridin-1-yl group (Dvas);
a 4-(2-imidazolyl)-pyrrolo[2,3-b]pyridin-1-yl group (dDia); and
a Ds derivative: 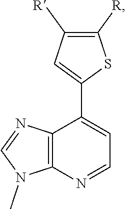
wherein R and R′ each independently represent any moiety represented by the following formula: 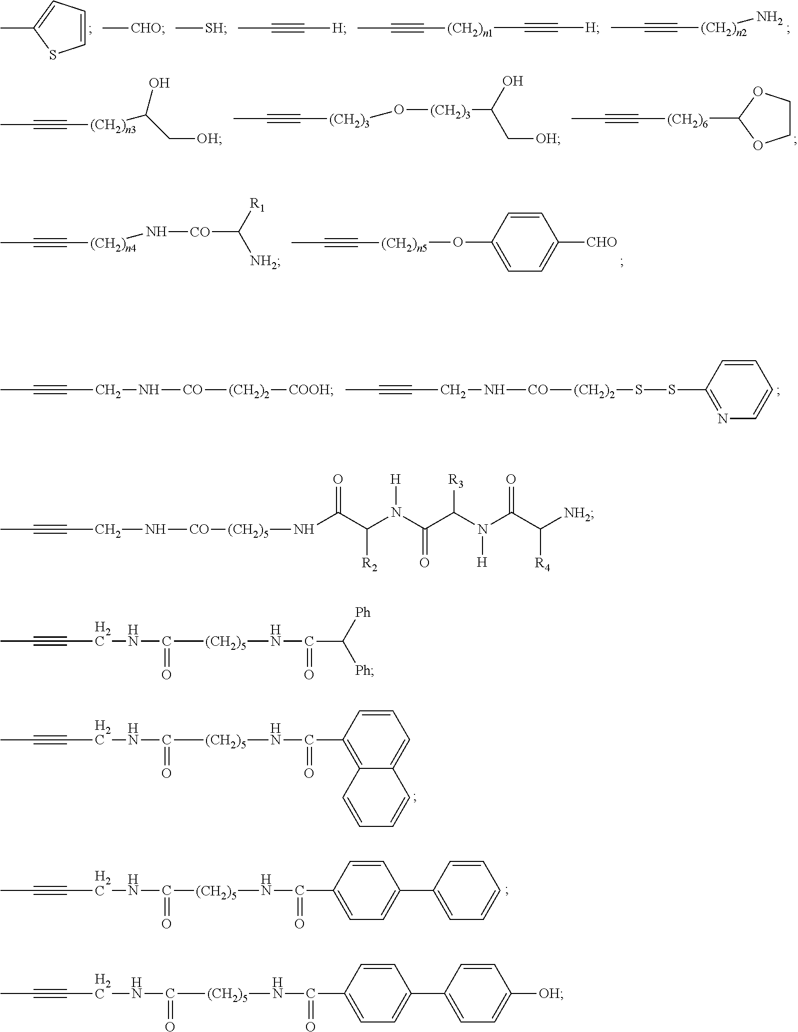
wherein n1=2 to 10; n2=1 or 3; n3=1, 6, or 9; n4=1 or 3; n5=3 or 6; R1=Phe (phenylalanine), Tyr (tyrosine), Trp (tryptophan), His (histidine), Ser (serine), or Lys (lysine); and R2, R3, and R4=Leu (leucine), Leu, and Leu, respectively, or Trp, Phe, and Pro (proline), respectively.

8. The method of claim 1, wherein the natural base pair is composed of a nucleobase selected from the group consisting of A, G, C, U, and T.

9. The method of claim 1, wherein the nucleic acid is a DNA strand.

10. The method of claim 1, wherein the library of pre-determined conversion rate comprises a ratio of the conversion of an unnatural base pair to either one of a natural base pair.

11. The method of claim 1, wherein the library of pre-determined conversion rate comprises a ratio of the conversion of an unnatural base pair to either one of a natural base pair based on the sequence of one or more adjacent base pair.

12. The method of claim 1, wherein the replacement replication reaction further comprises replicating the nucleic acid using natural base pairs.

13. The method of claim 1, wherein the replacement replication reaction is a replacement polymerase chain reaction (PCR).

14. The method of claim 1, wherein the replacement replication reaction comprises:
performing a first nucleic acid replication reaction using a first replication substrate containing an intermediate of the unnatural base pair to thereby replace the unnatural base pair with the intermediate of the unnatural base pair; and
performing a second nucleic acid replication reaction using a second replication substrate containing natural base pair to thereby replace the intermediate of the unnatural base pair with a natural base pair, optionally the replacement replication reaction further comprises:
replicating or amplification of the nucleic acid from the second nucleic acid replication reaction to thereby have a plurality of nucleic acid with natural base pair resulting from the second nucleic acid replication reaction.

15. (canceled)

16. The method of claim 1, wherein the sequencing is performed using deep sequencing method.

17. The method of claim 1, wherein the identifying the candidate position of the unnatural base pair comprises aligning the sequenced nucleic acid and determining a position that contains varying nucleobase.

18. The method of claim 1, wherein the ratio of conversion of the intermediate to each one of a natural base pair at the candidate position of the unnatural base pair is calculated using the formula:
% rA (at position i)=CR(A,i)=S(A,i)/[S(A,i)+S(G,i)+S(C,i)+S(T,i)]×100
where S(n, i) is the read numbers of sequences which has natural base n at position i.

19. The method of claim 1, wherein the substantial match of the ratio of conversion of the intermediate is a value that is within about 10% of the value in the library of the pre-determined conversion rate.

20. An apparatus for performing the method of claim 1.


SEQUENCE LISTING (11 sequences)

SEQ ID NO: 1 - DNA, 49 bp, Artificial Sequence
  DNA aptamer
  misc_feature (23)..(24): n is a, c, g, or t
  misc_feature (25)..(25): 7-(2-thienyl)-imidazo[4,5-b]pyridine
  misc_feature (26)..(27): n is a, c, g, or t
tagcgcataggtgggatgatgtnnnnngtcagatacaatcctgatccat

SEQ ID NO: 2 - DNA, 49 bp, Artificial Sequence
  DNA aptamer
  misc_feature (22)..(24): n is a, c, g, or t
  misc_feature (25)..(25): 7-(2-thienyl)-imidazo[4,5-b]pyridine
  misc_feature (26)..(28): n is a, c, g, or t
atcctcaccgatgtactgatgnnnnnnntcagatacaatcctgatccat

SEQ ID NO: 3 - DNA, 25 bp, Artificial Sequence
  DNA primer
ctatcactagcgcataggtgggatg

SEQ ID NO: 4 - DNA, 25 bp, Artificial Sequence
  DNA primer
agtctccatcctcaccgatgtactg

SEQ ID NO: 5 - DNA, 25 bp, Artificial Sequence
  DNA primer
ccgtctcatggatcaggattgtatc

SEQ ID NO: 6 - DNA, 192 bp, Artificial Sequence
  DNA aptamer
  misc_feature (26)..(26): natural-base tag sequence
  misc_feature (27)..(69): n is a, c, g, or t
  misc_feature (122)..(122): natural-base tag sequence
  misc_feature (123)..(165): n is a, c, g, or t
ctgtcaatcgatcgtatcagtccacnnnnnnnnnnnnnnnnnnnnnnnnnnnnnnnnnnnnnnnnnnnngcatgactcgaacggattagtgactacctgtcaatcgatcgtatcagtccacnnnnnnnnnnnnnnnnnnnnnnnnnnnnnnnnnnnnnnnnnnnngcatgactcgaacggattagtgactac

SEQ ID NO: 7 - DNA, 27 bp, Artificial Sequence
  DNA primer
ttctgtcaatcgatcgtatcagtccac

SEQ ID NO: 8 - DNA, 29 bp, Artificial Sequence
  DNA primer
aagtagtcactaatccgttcgagtcatgc

SEQ ID NO: 9 - DNA, 86 bp, Artificial Sequence
  DNA aptamer
  misc_feature (29)..(58): n is a, c, g, t, or 7-(2-thienyl)-imidazo[4,5-b]pyridine
atccgccatacttacgttgtccgtgaccnnnnnnnnnnnnnnnnnnnnnnnnnnnnnnggtcacgttggaatcttaagtgaagtcg

SEQ ID NO: 10 - DNA, 25 bp, Artificial Sequence
  DNA primer
tatatccgccatacttacgttgtcc

SEQ ID NO: 11 - DNA, 25 bp, Artificial Sequence
  DNA primer
gcgcgacttcacttaagattccaac